(19) United States
(12) Patent Application Publication
(10) Pub. No.: US 2023/0060581 A1
(43) Pub. Date: Mar. 2, 2023

(54) METHOD OF TREATING PANCREATIC CANCER

(71) Applicant: CEDARS-SINAI MEDICAL CENTER, Los Angeles, CA (US)

(72) Inventor: Andrew Hendifar, Santa Monica, CA (US)

(73) Assignee: CEDARS-SINAI MEDICAL CENTER, Los Angeles, CA (US)

(21) Appl. No.: 17/796,436

(22) PCT Filed: Feb. 9, 2021
(86) PCT No.: PCT/US2021/017286

§ 371 Date: Jul. 29, 2022

Related U.S. Application Data
(60) Provisional application No. 62/972,345, filed on Feb. 10, 2020.

(51) Int. Cl. A61K 31/519; A61K 31/4523; A61K 31/4184; A61K 31/166; A61K 31/506; A61K 31/4375; A61K 31/4155; A61K 31/196; A61K 31/365; A61K 39/395; A61K 31/4365; A61K 31/437; A61K 31/44; A61K 31/4706; A61K 31/282; A61K 31/337; A61P 35/00

(52) U.S. Cl. CPC A61K 31/519; A61K 31/4523; A61K 31/4184; A61K 31/166; A61K 31/506; A61K 31/4375; A61K 31/4155; A61K 31/196; A61K 31/365; A61K 39/3955; A61K 31/4365; A61K 31/437; A61K 31/44; A61K 31/4706; A61K 31/282; A61K 31/337; A61P 35/00

(57) ABSTRACT

The present invention provides for a method to treat pancreatic cancer. In various embodiments the method provides for administering one or more MAPK one or more MAPK pathway inhibitors to the subject, wherein the subject has a mutation in one or more genes in the MAPK signaling pathway.

### CROSS-REFERENCE TO RELATED APPLICATIONS

This application includes a claim of priority under 35 U.S.C. § 119(e) to U.S. provisional patent application No. 62/972,345, filed Feb. 10, 2020, the entirety of which is hereby incorporated by reference.

### FIELD OF INVENTION

This invention relates to treatment of pancreatic cancer.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Pancreatic cancer (PC) has a 5-year survival of 9% and is projected to be the second leading cause of cancer-related mortality in the United States before 2030. FDA approved therapies specifically for PC are limited to combinatorial cytotoxic regimens including FOLFIRINOX, gemcitabine with nab-paclitaxel, and nanoliposomal irinotecan with 5-fluorouracil. Despite the advent of precision medicine, olaparib as maintenance therapy in germline BRCA-mutated patients is the sole FDA approved targeted therapeutic. In addition, two classes of tumor agnostic targeted therapies include pancreatic cancer. PD-1 inhibitors have been approved for MSI-high or MMR deficient solid tumors, however, these types of tumors represent approximately 0.5-1% of pancreatic cancers. NTRK inhibitors are also FDA approved for TRK gene fusions, which are very rare in pancreatic cancer. As such, there remains an urgent need in the art for treatments for pancreatic cancer.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of treating pancreatic cancer in a subject in need thereof, comprising administering one or more MAPK pathway inhibitors to the subject, wherein the subject has a mutation in one or more genes in the MAPK signaling pathway.

In various embodiments, the subject can be KRAS wild type.

In various embodiments, the mutation can be in BRAF. In various embodiments, the mutation can be selected from: (a) BRAF V600E, (b) oncogenic fusion of BRAF and another gene, (c) non-V600 mutation, insertion or deletion, or (d) a mutation other than (a)-(c) with an additional driver mutation. In various embodiments, the mutation is BRAF N486 P490del.

In various embodiments, the subject can have a mutation in BRAF, and the one or more MAPK pathway inhibitors is a BRAF inhibitor, a MEK inhibitor or both.

In various embodiments, the one or more MAPK pathway inhibitors can be a MEK inhibitor. In various embodiments, the MEK inhibitor can be trametinib, cobimetinib, binimetinib, selumetinib, or mirdametinib. In various embodiments, the MEK inhibitor is Cobimetinib, Binimetinib, or Trametinib. In various embodiments, one or more MAPK pathway inhibitors can be dabrafenib mesylate and trametinib dimethyl sulfoxide, cobimetinib fumarate, binimetinib, binimetinib and encorafenib, trametinib dimethyl sulfoxide, selumetinib sulfate, LNP-3794, dabrafenib mesylate, panitumumab and trametinib dimethyl sulfoxide, HL-085, mirdametinib, MK-2206 and selumetinib sulfate, trametinib dimethyl sulfoxide and uprosertib, ATI-450, ATR-002, CKI-27, CS-3006, durvalumab and selumetinib sulfate, E-6201, FCN-159, SHR-7390, TQB-3234, ABM-1383, ATR-004, ATR-005, ATR-006, EBI-1051, KZ-001, OTS-514, OTS-964, Small Molecule to Inhibit MKK4 for Acute Liver Failure and Chronic Liver Disease, Small Molecules to Inhibit Mitogen Activated Protein Kinase Kinase for Oncology, or SNR-1611.

In various embodiments, the one or more MAPK pathway inhibitors can be a BRAF inhibitor. In various embodiments, the BRAF inhibitor is dabrafenib mesylate and trametinib dimethyl sulfoxide, sorafenib tosylate, binimetinib and encorafenib, dabrafenib mesylate, encorafenib, vemurafenib, sorafenib tosylate, dabrafenib mesylate, panitumumab and trametinib dimethyl sulfoxide, hydroxychloroquine and sorafenib tosylate, lifirafenib maleate, TAK-580, BAL-3833, belvarafenib, CKI-27, LUT-014, LXH-254, RXDX-105, TQB-3233, XP-102, UB-941, ABM-1310, AFX-1251, APL-102, ARI-4175, AZ-304, BGB-3245, INU-152, LYN-204, PV-103, REDX-05358, or SJP-1601.

In various embodiments, the BRAF inhibitor can be Dabrafenib, Vemurafenib, Encorafenib, Lifirafenib, belvarafenib, or sorafenib tosylate. In various embodiments, the BRAF inhibitor can be Vemurafenib, Dabrafenib or Encorafenib, or combinations thereof.

In various embodiments, the one or more MAPK pathway inhibitors can be Dabrafenib and trametinib, Vemurafenib and cobimetinib, Trametinib, or Vemurafenib. In various embodiments, the one or more MAPK pathway inhibitor can be Binimetinib and Encorafenib. In various embodiments, the one or more MAPK pathway inhibitors can be Vemurafenib and the method further comprises administering carboplatin, paclitaxel or both. In various embodiments, the one or more MAPK pathway inhibitors can be Trametinib and the method further comprises administering Pembrolizumab.

In various embodiments, the method can further comprise administering one or more chemotherapy drugs, one or more PD-1 inhibitors or PD-L1 inhibitors, both a chemotherapy drug and a PD-1 inhibitor, or both a chemotherapy drug and a PD-L1 inhibitor.

In various embodiments, the one or more MAPK pathway inhibitor can be Binimetinib and Encorafenib, wherein the mutation is BRAF V600E, and the pancreatic cancer is BRAF V600E mutated pancreatic ductal adenocarcinoma (PDAC).

In various embodiments, the subject's pancreatic cancer has progressed on at least one line of therapy for metastatic disease or wherein the subject can be intolerant of at least one line of therapy for metastatic disease.

In various embodiments, the subject's pancreatic cancer has recurred with metastatic disease less than or equal to 12 weeks of completion of neoadjuvant or adjuvant systemic chemotherapy, or wherein the subject has locally advanced pancreatic cancer whose pancreatic cancer progressed to metastatic disease less than or equal to 12 weeks after completion of systemic chemotherapy, or wherein the subject's pancreatic cancer has recurred with metastatic disease less than or equal to 12 weeks of completion of systemic chemotherapy.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A and 1B depict genomic profiling results from pancreatic tumors harboring BRAF pathway alterations. 1A) Lollipop plot highlighting amino acid positions along the BRAF gene where alterations were most commonly found in this case series (n=81). Each stemmed circle represents the numbers of patients with a BRAF alteration at each position (or type for structural variants), counted separately based on either the presence (downward lollipop) or absence (upward lollipop) of a confounding alteration in another oncogenic driver (e.g. KRAS mutation). BRAF-altered molecular profiles were categorized into four subgroups that have been associated with distinct implications for therapy: Exon 15 (red; V600 mutations that have been associated with responsiveness to canonical BRAF inhibitors); Exon 11 (orange; non-V600 mutations that confer RAS-independent activity but are likely vemurafenib-insensitive); Fusions (purple; intergenic structural variants); and Other (blue; structural and/or short variants, either uncharacterized, characterized as RAS-dependent mutations, or found alongside confounding driver mutations). The three most common variants (BRAF V600E, BRAF N486_P490del also known as ΔNVTAP, and SND1-BRAF fusions) are highlighted at 3 hotspots that form the basis for the subgroups. 1B) Molecular matrix organized by BRAF subgroup shows genomic testing results for each patient including specific BRAF variants, confounding drivers, and p53/CDKN2A/SMAD4 mutations.

FIG. 2 depicts overall survival of advanced PC subjects by BRAF subgroup. No significant differences in overall survival (from initial diagnosis) were observed across these four categories (p>0.05, pairwise comparisons evaluated by Cox regression) suggesting that these functional classifications of BRAF alterations are not likely prognostic. For this survival analysis, patients diagnosed with IPMN's and resected disease were excluded (see “OS Cohort” in Table 2 for additional baseline characteristics).

FIG. 3 depicts progression-free survival data while on BRAF/MEK/ERK inhibitors for patients with BRAF-mutated pancreatic cancer. Responses to 17 patients treated with BRAF-directed therapy categorized by BRAF subgroup, including 6 patients treated with combination BRAF and MEK and 8 patients with MEK inhibitor therapy alone. Each horizontal bar represents the time on therapy without disease progression (i.e. progression-free survival). Darker green represents treatment lines with partial response as best response, light green are patients who achieved stable disease, copper bars are patients with rapidly progressive disease (no response), and white bars indicate unevaluable subjects who had discontinued due to tolerability issues (censored event). Bars capped with arrows indicate lines of therapy that were continuing as of the last available progress note (censored event).

FIGS. 4A-4D depicts progression-free survival analysis across BRAF classes for two types of standard therapies commonly implemented in pancreatic cancer. PFS while receiving either 1^{st} line FOLFIRINOX (4A), 5FU-based chemotherapy (e.g. FOLFIRINOX or 5-Fluorouracil/nal-Irinotecan) in 2^{nd} line or later (4B), or gemcitabine/nab-paclitaxel given in 1^{st} line (4C) or later lines (4D) were analyzed for each of the four categories of BRAF alterations with median PFS values [95% confidence intervals] shown. Abbreviations: N/R (not reached); N/A (not applicable)

FIGS. 5A-5D depict overall survival analysis comparing patients who received a molecularly-matched therapy targeting the MAPK signaling pathway (e.g. BRAF/MEK/ERK inhibitors) versus those who only received unmatched therapies in the advanced treatment setting. Overall survival differences between matched and unmatched subgroups were not considered statistically significant for either BRAF subgroups Exon 15 (5A), Exon 11 (5B), Fusions (5C), or Other (5D) alterations when analyzed individually (p>0.05). For the broader subset of patients, mOS differences were trending toward benefit but not considered significant (p=0.07252 (HR=0.48 [0.21-1.07])) when comparing matched (mOS=1.92y [1.37-N/A], n=14) and unmatched (mOS=1.51y [0.95-2.89], n=25) subgroups. Only patients who were initially diagnosed with metastatic disease were included in these analyses (see “OS Matched” and “OS Unmatched” subgroups in Table 2 for additional baseline characteristics across the combined cohort).

FIGS. 6A-6B depict PFS analyses highlighting favorable trends for 5FU-based therapies versus gemcitabine/nab-paclitaxel in patients with RAF fusions. A significant difference in mPFS was observed for FOLFIRINOX versus gemcitabine/nab-paclitaxel within the BRAF Fusion subgroup (6A) using a univariate Cox regression model across all lines of therapy (p=0.0051 (HR=0.1 [0.02-0.50] or (6B) using a multivariate model (p=0.027) that factored in therapies given in 1^{st} line of therapy versus later lines. While these trends were considered significant, prospective evaluation is warranted when considering the imbalance between treatment choices for 1^{st} line, an unexpected trend of longer PFS for later lines versus P^{t} line (note: this term was not significant in the multivariate model), the relatively small sample sizes, among other potentially confounding factors. Within this subset of the BRAF fusion analysis cohort, acinar cell carcinoma histology was seen in 5 (36% of 14) and 6 (50% of 12) for 5FU-based vs Gemcitabine-based therapies, respectively (the rest were adenocarcinoma). This variable was not significantly enriched by Fisher's exact test (p=0.69) and its addition to the multivariate Cox regression model yielded similar results for the contrast between regimens (p=0.0405, HR=0.1 [0.01-0.9]).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal may be a human, non-human primate, mouse, rat, dog, cat, horse, or cow, but are not limited to these examples. In addition, the methods described herein may be used to treat domesticated animals and/or pets. In some embodiments, the subject is a human. In some embodiments, the subject is a male subject. In some embodiments, the subject has a cancer. In some embodiments, the subject has a tumor.

A subject may be one who has been previously diagnosed with or identified as suffering from or having a disease, disorder or condition in need of treatment or one or more complications related to the disease, disorder, or condition, and optionally, have already undergone treatment for the disease, disorder, or condition or the one or more complications related to the disease, disorder, or condition. Alternatively, a subject can also be one who has not been previously diagnosed as having a disease, disorder, or condition or one or more complications related to the disease, disorder, or condition. For example, a subject may be one who exhibits one or more risk factors for a disease, disorder, or condition or one or more complications related to the disease, disorder, or condition or a subject who does not exhibit risk factors. A “subject in need” of treatment for a particular disease, disorder, or condition may be a subject suspected of having that disease, disorder, or condition, diagnosed as having that disease, disorder, or condition, already treated or being treated for that disease, disorder, or condition, not treated for that disease, disorder, or condition, or at risk of developing that disease, disorder, or condition.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be including within the scope of this term.

“Therapeutically effective amount” as used herein refers to that amount which is capable of achieving beneficial results in a patient with pancreatic cancer. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or lessen the disease even if the treatment is ultimately unsuccessful.

“Disease progression” as used herein refers to clinical or radiographic disease progression, as defined by RECIST 1.1 criteria.

“Progression-free survival” (PFS) as used herein refers to the time from the first administration of the inventive therapy described herein to the first of either disease progression or death from any cause, where disease progression is determined based on RECIST 1.1 criteria. PFS will be estimated using the Kaplan-Meier method. The median PFS and corresponding 95% confidence interval will be reported. Patients who do not experience disease progression or death while on protocol will be censored at the last disease assessment date.

“Overall survival” (OS) as used herein refers to the time from the first administration of the inventive therapy described herein to death from any cause.

“Duration of response” as used herein refers to the duration of time from first documentation of an objective response to the earliest date disease progression is documented or death from any cause.

“Time to response” as used herein refers to the duration of time from the first administration of the inventive therapy described herein to the first documentation of an objective response.

PC sequencing efforts by the International Cancer Genome Consortium (ICGC), The Cancer Genome Atlas (TCGA) and others have identified a preponderance of KRAS mutations (92-93%). In those lacking KRAS alterations, driver mutations have been identified including: BRAF, GNAS, CTNNB1, ROS, NRG1, and ALK fusions. BRAF mutations have been previously identified in KRAS wild-type (WT) and mutant pancreatic cancer cell lines and patient tumors.

Alterations in BRAF are classified into 3 functional groups based on the dependence of RAS activation, BRAF kinase activity, and signaling properties. Class I BRAF mutations result in RAS-independent kinase activation and signal as monomers, e.g., BRAF V600E. Class II mutations are also RAS-independent but signal as heterodimers and rarely co-occur with other alterations in the MAPK pathway. This group includes activating BRAF mutations, fusions, and in-frame deletions. Class III mutations are RAS dependent and occur when they heterodimerize with wild-type CRAF and amplify RAS signaling, often co-occurring with activating RAS or NF1 loss-of-function mutations.

Beyond PC, recurrent BRAF alterations are observed across multiple cancer types including lung, colon, thyroid, and melanoma, as well as in non-Hodgkin lymphoma. In pancreatic acinar cell carcinomas, rearrangements involving BRAF and RAF1 (CRAF) have been identified in approximately 23% of tumors. Combined BRAF and MEK inhibition is now FDA approved for metastatic melanoma, non-small cell lung cancer and anaplastic thyroid cancer. In addition, targeting BRAF V600 alterations with combinations of RAF/MEK inhibition and anti-EGFR therapy has improved outcomes in colorectal cancer and received breakthrough status in 2018.

Commercial and in-house Next Generation Sequencing (NGS) platforms are providing precision medicine in PC. These efforts have identified several potential targets for development. However, there remains a lack of functional studies and outcome data in BRAF mutated PC.

As described herein we address this knowledge gap by examining all reported BRAF alterations in epithelial pancreatic malignancies. To provide clinical support for genomic and preclinical BRAF predictions and therapeutic strategies we have compiled the largest case series of BRAF altered PC, and report outcomes and responses to BRAF directed therapy.

Activation of the RAS/RAF/MEK/ERK pathway is critical for the proliferation, survival, and tumorigenesis in approximately 30% of all malignancies and 90-95% of all pancreatic malignancies. To date, only oncogenic mutations in KRAS have been well characterized for PC. We demonstrate herein that BRAF alterations have biological and clinical relevance in the KRAS WT population. Utilizing large published datasets, and a multi-institutional retrospective case series, we report the first comprehensive evaluation of BRAF alterations in pancreatic cancer, associated clinical outcomes, and evaluate response to BRAF-directed therapy.

Previous studies have identified driver BRAF mutations in KRAS wild-type pancreatic cancer. In our review of all publicly available sequencing PC data we have confirmed BRAF alterations occurred in 2% of pancreatic ductal adenocarcinoma and 25% of pancreatic acinar cell tumors. These alterations in PDAC occurred evenly between Exon 15 (V600), fusions, Exon 11(non-V600), and non-canonical alterations. Exon 15 (V600) alterations were also identified for the first time in a pancreaticoblastoma and a pseudopapillary neoplasm of the pancreas.

We then compiled the largest retrospective series of PC patients with BRAF alterations to date. We confirm that pancreatic cancer is characterized by a unique spectrum of non-exon 15 (V600) variants in the BRAF gene including a recurring five-amino-acid (ΔNVTAP) deletion in the BRAF β3-αC loop (i.e. BRAFA^{ΔNvTAP}), and BRAF gene fusions.

In our 81-case cohort, exon 15 and exon 11 mutations, and fusion events were found to be almost entirely exclusive of KRAS alterations (62/63). In individuals with missense mutations, categorized as non-canonical, 11/19 had KRAS mutations. Our findings indicate that BRAF alterations are an important driver in many KRAS WT pancreatic tumors.

In this cohort we confirm reports of clinical responses to MEK and BRAF inhibition in subjects with biologically significant BRAF alterations. Benefit was well aligned with the classification system described by Yaeger et al., as many subjects with class 1 (V600E) and 2 (fusion) abnormalities had enhanced responses. Whereas, we report no responses in individuals with other abnormalities, some of which are considered class 3 variants or were poorly-characterized alterations that were reported as pathogenic by the genomic testing laboratory.

Many of the BRAF alterations found in this cohort have not been fully characterized and were not able to be placed in this functional classification system. For example, BRAF T599 V600insT is a change in the amino acid sequence of the serine/threonine protein kinase B-raf protein where a threonine residue has been inserted between the threonine at position 599 and the valine at position 600. In-vitro experiments have demonstrated high kinase activity and dimerization dependence. However, there are clinical reports of responsiveness to BRAF inhibition in a patient with metastatic melanoma. The BRAF ΔNVTAP deletion was unusually prevalent in our cohort of BRAF alterations 16/81. This short, in-frame 5 amino acid deletion within the kinase domain (β3-αC Loop) which regulates BRAF kinase activity. These alterations result in increased kinase activity but are resistant to the BRAF inhibitor vemurafenib. However, there are clinical case reports of significant activity with dabrafenib in patients with ΔNVTAP deletion which aligns with the observation that dabrafenib fits better than vemurafenib inside the BRAF pocket. In addition, many of the fusions and missense mutations presented here have not been fully characterized. These include: D566E; E26D; E501K; G596R; K601N; N236K; P403Lfs*8; Q94K; R389H; S467L; T241P; T310I; and truncation intron 8.

In our cohort, only individuals receiving approved MEK inhibitors or approved combinations of MEK and BRAF inhibitors had clinical benefit. In a recently published study, 3 patients with pancreatic cancer were enrolled in a 172 patient pan-cancer BRAF V600 study and results were consistent with our findings. Individuals who had received BRAF-directed therapy demonstrated a trend toward improved outcomes vs. those who did not. Notably these BRAF alterations occur across a spectrum of epithelial pancreatic tumors underscoring the importance of routine molecular profiling irrespective of histology across pancreatic cancers, particularly acinar cell carcinomas (which commonly harbor BRAF fusions) and other pancreaticobiliary tumors (e.g. cholangiocarcinoma, ampullary, and duodenal carcinomas).

We examined BRAF categorization as a prognostic or predictive factor. In our cohort, BRAF categorization was not associated with differences in overall survival. Unlike previous reports in colon cancer, BRAF V600E alterations were not predictive of poor response to chemotherapy. However, we found that BRAF fusion abnormalities may speculatively represent a predictive marker of improved response to FOLFIRINOX and poor response to gemcitabine and nab-paclitaxel. We were unable to find any evaluation of chemotherapeutic response to tumors harboring fusion abnormalities outside of pemetrexed therapy in lung cancers with ROS1 fusion abnormalities.

Due to the high unmet need in the PC patient population and the infrequency of BRAF alterations, a single arm prospective trial confirming substantial response rates and durability of responses would likely be sufficient to convince the FDA to extend the indication labelling of the previously FDA approved BRAF and MEK inhibitors to include patients with BRAF-mutated PC.

Herein we have described a cohort of BRAF-mutated pancreatic cancer that comprises 2% of PC cases and can be classified into 4 functional categories. We report promising treatment responses and encouraging outcomes in patients receiving BRAF-directed therapy. These responses were functional class dependent and occurred in Class 1 and 2 abnormalities.

Embodiments of the present invention, are based at least in part, on these findings.

Various embodiments of the present invention provide for a method of treating pancreatic cancer in a subject in need thereof, comprising administering one or more MAPK pathway inhibitors to the subject, wherein the subject has a mutation in one or more genes in the MAPK signaling pathway.

In various embodiments, the subject has been tested and determined to have the mutation in one or more genes in the MAPK signaling pathway. In various embodiments, the method further comprises testing the subject for a mutation in one or more genes in the MAPK signaling pathway prior to administering the one or more MAPK pathway inhibitors to the subject. In various embodiments, the subject's cancer has a mutation in one or more genes in the MAPK signaling pathway. In various embodiments, the pancreatic cancer comprises the mutation in one or more genes in the MAPK signaling pathway.

In various embodiments, administering one or more MAPK pathway inhibitors to the subject imparts a duration of response that is longer as compared to standard of care therapy available at the time of the present invention. In various embodiments, administering one or more MAPK pathway inhibitors to the subject imparts a progression free survival that is longer as compared to standard of care therapy available at the time of the present invention. In various embodiments, administering one or more MAPK pathway inhibitors to the subject imparts an overall survival that is longer as compared to standard of care therapy available at the time of the present invention.

In various embodiments, the subject's pancreatic cancer has progressed on at least one line of therapy for metastatic disease or wherein the subject is intolerant of at least one line of therapy for metastatic disease. In various embodiments, the subject's pancreatic cancer has recurred with metastatic disease less than or equal to 12 weeks of completion of neoadjuvant or adjuvant systemic chemotherapy. In various embodiments, wherein the subject has locally advanced pancreatic cancer whose pancreatic cancer progressed to metastatic disease less than or equal to 12 weeks after completion of systemic chemotherapy. In various embodiments, the subject's pancreatic cancer has recurred with metastatic disease less than or equal to 12 weeks of completion of systemic chemotherapy.

In various embodiments, the pancreatic cancer is adenocarcinoma, acinar cell carcinoma, mixed acinar/neuroendocrine carcinoma, or solid pseudopapillary neoplasm.

In various embodiments, the mutation is in BRAF. In various embodiments, the mutation is selected from: (a) BRAF V600E, (b) oncogenic fusion of BRAF and another gene, (c) non-V600 mutation, insertion or deletion, or a mutation other than (a)-(c) with an additional driver mutation.

In various embodiments, the mutation is BRAF V600E. In various embodiments, the mutation is BRAF N486_P490del.

In various embodiments, the mutation is on exon 15. In various embodiments, the mutation is V600E, V600_K601delinsE, T599dup, V600R, or V600E and Q609L.

In various embodiments, the mutation is on exon 11. In various embodiments, the mutation is G469A, K483E, L485F, or V487_P492delinsA.

In various embodiments, the mutation is a fusion mutation. In various embodiments, the mutation is SND1-BRAF Fusion, BRK1-RAF1 Fusion, DTNA-RAF1 Fusion, FGD5-RAF1 Fusion, GIPC2-BRAF Fusion, GLI2-BRAF Fusion, JHDM1D-BRAF Fusion; LUC7L2-BRAF Fusion, MKRN1-BRAF Fusion, TMEM9-BRAF Fusion, RAF1 Rearrangement, or BRAF Rearrangement.

In various embodiments, the mutation is T310I, D594G, G469S, G596D, G596R, N236K, S467L, T241P, X327_splice, ARAF S214A, RAF1 S259Y, T599K, V600 and a Confounding Driver, or N486_P490del and ROS1 Fusion.

In various embodiments, the subject has a mutation in BRAF, and the one or more MAPK pathway inhibitors is a BRAF inhibitor, a MEK inhibitor or both. In various embodiments, the subject has KRAS wild type.

In various embodiments, the one or more MAPK pathway inhibitors is a MEK inhibitor. In various embodiments, the MEK inhibitor is trametinib, cobimetinib, binimetinib, selumetinib, or mirdametinib. In various embodiments, the MEK inhibitor is Cobimetinib, Binimetinib, or Trametinib.

In various embodiments, the one or more MAPK pathway inhibitors is dabrafenib mesylate and trametinib dimethyl sulfoxide, cobimetinib fumarate, binimetinib, binimetinib and encorafenib, trametinib dimethyl sulfoxide, selumetinib sulfate, LNP-3794, dabrafenib mesylate, panitumumab and trametinib dimethyl sulfoxide, HL-085, mirdametinib, MK-2206 and selumetinib sulfate, trametinib dimethyl sulfoxide and uprosertib, ATI-450, ATR-002, CKI-27, CS-3006, durvalumab and selumetinib sulfate, E-6201, FCN-159, SHR-7390, TQB-3234, ABM-1383, ATR-004, ATR-005, ATR-006, EBI-1051, KZ-001, OTS-514, OTS-964, Small Molecule to Inhibit MKK4 for Acute Liver Failure and Chronic Liver Disease, Small Molecules to Inhibit Mitogen Activated Protein Kinase for Oncology, or SNR-1611.

In various embodiments, the one or more MAPK pathway inhibitors is a BRAF inhibitor. In various embodiments, the BRAF inhibitor is dabrafenib mesylate and trametinib dimethyl sulfoxide, sorafenib tosylate, binimetinib and encorafenib, dabrafenib mesylate, encorafenib, vemurafenib, sorafenib tosylate, dabrafenib mesylate, panitumumab and trametinib dimethyl sulfoxide, hydroxychloroquine and sorafenib tosylate, lifirafenib maleate, TAK-580, BAL-3833, belvarafenib, CKI-27, LUT-014, LXH-254, RXDX-105, TQB-3233, XP-102, UB-941, ABM-1310, AFX-1251, APL-102, ARI-4175, AZ-304, BGB-3245, INU-152, LYN-204, PV-103, REDX-05358, or SJP-1601. In various embodiments, the BRAF inhibitor is Dabrafenib, Vemurafenib, Encorafenib, Lifirafenib, belvarafenib, or sorafenib tosylate. In various embodiments, the BRAF inhibitor is Vemurafenib, Dabrafenib and Encorafenib.

In various embodiments, the one or more MAPK pathway inhibitors is Dabrafenib and trametinib, Vemurafenib and cobimetinib, Trametinib, or Vemurafenib. In various embodiments, the one or more MAPK pathway inhibitor is Binimetinib and Encorafenib. In various embodiments, the one or more MAPK pathway inhibitors is Vemurafenib and the method further comprises administering carboplatin and paclitaxel. In various embodiments, the one or more MAPK pathway inhibitors is Trametinib and the method further comprises administering Pembrolizumab.

In various embodiments, administering one or more MAPK pathway inhibitors to the subject comprises one or more cycles. In various embodiments, administering one or more MAPK pathway inhibitors to the subject comprises 2, 3, 4, 5, 6, 7, 8, 9, or 10 or more cycles. In various embodiments, each cycle is about 28 days. In various embodiments, each cycle is about 21 days. In various embodiments, each cycle is about 35 days. In various embodiments, each cycle is about 7, 14, 21, 28, 35, 42, 49 or 56 days.

In various embodiments, the method comprises administering up to 36 cycles. In other embodiments, the method comprises administering more than 36 cycles. In various embodiments, the method comprises administering 1-3 cycles, 4-6 cycles, 7-9 cycles, 10-12 cycles, 13-15 cycles, 16-18 cycles, 19-21 cycles, 22-24 cycles, 25-27 cycles, 28-30 cycles, 31-33 cycles, or 35-56 cycles. In various embodiments, the method comprises administering until disease progression.

In various embodiments cycle 2 is the same as cycle 1. In various embodiments, the doses for cycle 3 is less than cycle 1 or cycle 2. In various embodiments, the doses for cycle 3 and/or subsequent cycles is less than cycle 1 or cycle 2.

In various embodiments the doses can be reduced after one cycle. In various embodiments the doses can be reduced after two cycles. Reduction of doses can be due; for example, to adverse events as described herein.

In some embodiments of the invention, the dose of the one or more MAPK pathway inhibitors can each be in the range of about 10-50 μg/day, 50-100 μg/day, 100-150 μg/day, 150-200 μg/day, 100-200 μg/day, 200-300 μg/day, 300-400 μg/day, 400-500 μg/day, 500-600 μg/day, 600-700 μg/day, 700-800 μg/day, 800-900 μg/day, 900-1000 μg/day, 1000-1100 μg/day, 1100-1200 μg/day, 1200-1300 μg/day, 1300-1400 μg/day, 1400-1500 μg/day, 1500-1600 μg/day, 1600-1700 μg/day, 1700-1800 μg/day, 1800-1900 μg/day, 1900-2000 μg/day, 2000-2100m/day, 2100-2200 μg/day, 2200-2300 μg/day, 2300-2400 μg/day, 2400-2500 μg/day, 2500-2600 μg/day, 2600-2700m/day, 2700-2800 μg/day, 2800-2900m/day or 2900-3000 μg/day.

In various embodiments, the dose of the one or more MAPK pathway inhibitors can be can each be in the range of about 10-50 mg/day, 50-100 mg/day, 100-150 mg/day, 150-200 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day.

In various embodiments, the method further comprises administering one or more chemotherapy drugs, one or more PD-1 inhibitors or PD-L1 inhibitors, both a chemotherapy drug and a PD-1 inhibitor, or both a chemotherapy drug and a PD-L1 inhibitor.

Examples of chemotherapeutic agents include cytotoxic agents (e.g., 5-fluorouracil, cisplatin, carboplatin, methotrexate, daunorubicin, doxorubicin (Adriamycin®), vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic akylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Examples of anti-PD1 inhibitor that can be used in the methods described herein can be selected from the group consisting of pembrolizumab, nivolumab, pidilizumab, AMP-224, AMP-514, spartalizumab, cemiplimab, AK105, BCD-100, BI 754091, JS001, LZMO09, MGA012, Sym021, TSR-042, MGD013, AK104, XmAb20717, tislelizumab, PF-06801591, anti-PD1 antibody expressing pluripotent killer T lymphocytes (PIK-PD-1), autologous anti-EGFRvIII 4SCAR-IgT cells, and combinations thereof. Examples of anti-PDL1 inhibitor that can be used in the methods described herein can be selected from the group consisting of BGB-A333, CK-301, FAZ053, KN035, MDX-1105, MSB2311, SHR-1316, atezolizumab, avelumab, durvalumab, BMS-936559, CK-301, M7824, and combinations thereof.

In various embodiments, the method of treating pancreatic cancer in a subject in need thereof, comprising administering one or more MAPK pathway inhibitors to the subject, wherein the subject has a mutation in one or more genes in the MAPK signaling pathway, wherein the one or more MAPK pathway inhibitor is Binimetinib and Encorafenib, wherein the mutation is BRAF V600E, and the pancreatic cancer is BRAF V600E mutated pancreatic ductal adenocarcinoma (PDAC). In various embodiments, the subject's pancreatic cancer has progressed on at least one line of therapy for metastatic disease or wherein the subject is intolerant of at least one line of therapy for metastatic disease. In various embodiments, the subject's pancreatic cancer has recurred with metastatic disease less than or equal to 12 weeks of completion of neoadjuvant or adjuvant systemic chemotherapy. In various embodiments, wherein the subject has locally advanced pancreatic cancer whose pancreatic cancer progressed to metastatic disease less than or equal to 12 weeks after completion of systemic chemotherapy. In various embodiments, the subject's pancreatic cancer has recurred with metastatic disease less than or equal to 12 weeks of completion of systemic chemotherapy.

In various embodiments, administering one or more MAPK pathway inhibitors to the subject comprises one or more cycles. In various embodiments, administering one or more MAPK pathway inhibitors to the subject comprises 2, 3, 4, 5, 6, 7, 8, 9, or 10 or more cycles. In various embodiments, each cycle is about 28 days. In various embodiments, each cycle is about 21 days. In various embodiments, each cycle is about 35 days. In various embodiments, each cycle is about 7, 14, 21, 28, 35, 42, 49 or 56 days.

In various embodiments, the method comprises administering up to 36 cycles. In other embodiments, the method comprises administering more than 36 cycles. In various embodiments, the method comprises administering 1-3 cycles, 4-6 cycles, 7-9 cycles, 10-12 cycles, 13-15 cycles, 16-18 cycles, 19-21 cycles, 22-24 cycles, 25-27 cycles, 28-30 cycles, 31-33 cycles, or 35-56 cycles. In various embodiments, the method comprises administering until disease progression.

In various embodiments, each cycle comprises administering about 150-525 mg of encorafenib orally daily and about 7.5-52.5 mg of binimetinib orally twice daily; for example, for 28 days.

In various embodiments, each cycle comprises administering about 225-450 mg of encorafenib orally daily and about 15-45 mg of binimetinib orally twice daily; for example, for 28 days.

In various embodiments, each cycle comprises administering about 450 mg of encorafenib orally daily and about 45 mg of binimetinib orally twice daily; for example, for 28 days. In various embodiments, each cycle comprises administering about 300 mg of encorafenib orally daily and about 30 mg of binimetinib orally twice daily; for example, for 28 days. In various embodiments, each cycle comprises administering about 225 mg of encorafenib orally daily and about 15 mg of binimetinib orally twice daily; for example, for 28 days.

In various embodiments, cycle 1 comprises administering about 450 mg of encorafenib orally daily and about 45 mg of binimetinib orally twice daily. In various embodiments cycle 2 is the same as cycle 1. In various embodiments, the doses for cycle 3 is less than cycle 1 or cycle 2. In various embodiments, the doses for cycle 3 and/or subsequent cycles is less than cycle 1 or cycle 2.

In various embodiments, cycle 1 comprises administering about 300 mg of encorafenib orally daily and about 30 mg of binimetinib orally twice daily. In various embodiments cycle 2 is the same as cycle 1.

In various embodiments, cycle 1 comprises administering about 225 mg of encorafenib orally daily and about 15 mg of binimetinib orally twice daily. In various embodiments cycle 2 is the same as cycle 1.

In various embodiments the doses can be reduced after one cycle. In various embodiments the doses can be reduced after two cycles. Reduction of doses can be due; for example, to adverse events as described herein.

Reduced doses can comprise; for example, administering about 300 mg of encorafenib orally daily and about 30 mg of binimetinib orally twice daily; or administering about 225 mg of encorafenib orally daily and about 15 mg of binimetinib orally twice daily.

In various embodiments, doses of encorafenib is not re-escalated after the dose reduction; for example, due to prolonged ATcF ≥about 501 msec.

In various embodiments, doses of binimetinib is not allowed to be re-escalated; for example, after a dose reduction due to LVEF dysfunction.

In various embodiments, doses of binimetinib or encorafenib is not allowed to be re-escalated; for example, after a dose reduction due to retinal toxicity ≥Grade 2.

In various embodiments, if binimetinib is withheld, encorafenib can be reduced to a maximum dose of 300 mg daily until binimetinib is resumed.

As used herein, the term “administering,” refers to the placement an agent as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, pen-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrastemal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions may be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions may be in the form of aerosol, lotion, cream, gel, ointment, suspensions, solutions or emulsions. In accordance with the present disclosure, “administering” may be self-administering. For example, it is considered as “administering” that a subject consumes a composition as disclosed herein.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

BRAF Alteration Frequency in a Real-World Cohort with Genomic Testing Results

To assess the frequency of BRAF alterations in PC, real-world data were obtained via the Perthera Platform which includes 1802 patients who underwent molecular profiling as part of the Know Your Tumor Program (KYT) and other hospital programs. Additional public data were obtained from 1979 patients with genomic testing results available via the AACR GENIE project (release 6.1.0). Genomic profiling data from this aggregated cohort of 3781 patients with pancreatic cancer were analyzed to assess the prevalence of BRAF alterations (see the “Prevalence Cohort” described in Table 1). Molecular profiles with fewer than 3 genomic variants detected were removed from the aggregated prevalence cohort to exclude low quality genomic testing results. Given the deidentified nature of these two data sets, the possibility of subjects with duplicative entries cannot be entirely ruled out; however, spot checking for highly similar variant profiles across databases suggested minimal overlap (data not shown). Histologic subtypes included in our case series were epithelial pancreatic cancers including ductal adenocarcinoma, acinar cell carcinoma, solid pseudopapillary neoplasm, and pancreaticoblastoma. Tumors with predominantly neuroendocrine features were excluded from the clinical case series cohort.

TABLE 1
Overview of BRAF alterations identified pancreatic tumors via genomic profiling
across the Prevalence Cohort** and the Clinical Case Series Cohort*

| BRAF | Prevalence | Pancreatic Cancer | Clinical | Pancreatic Cancer |
|---|---|---|---|---|
| Alterations | Cohort | Subtypes/Alterations | Cohort | Subtypes/Alterations |
| All | 84/3781 | adenocarcinoma: | 81 | adenocarcinoma(62); acinar |
| | (2.2%) | 64/3298 (1.9%); acinar | | cell or mixed |
| | | cell carcinoma: 9/49 | | acinar/neuroendocrine(14); |
| | | (18.4%); mixed | | IPMN(4); |
| | | acinar/neuroendocrine: | | pancreaticoblastoma(1) |
| | | 8/316 (2.5%); IPMN: | | |
| | | 3/21 (14.3%); solid | | |
| | | pseudopapillary | | |
| | | neoplasm: 1/16 (6.2%) | | |
| Exon 15 | 26/84 | V600E(16); | 17/81 | V600E(12); T599dup(3); |
| (V600) | (31.0%) | V600_K601delinsE(4); | (21.0%) | V600_K601delinsE; V600E |
| | | T599dup(4); V600R; | | & Q609L |
| | | V600E & Q609L | | |
| Exon 11 | 21/84 | N486_P490del(16); | 18/81 | N486_P490del(16); G469A; |
| (non-V600) | (25.0%) | G469A(2); K483E; | (22.2%) | V487_P492delinsA |
| | | L485F; | | |
| | | V487_P492delinsA | | |
| Fusion | 20/84 | SND1-BRAF Fusion(9); | 25/81 | SND1-BRAF Fusion(12); |
| | (23.8%) | BRK1-RAF1 Fusion; | (30.9%) | EPS15L1-BRAF Fusion; |
| | | DTNA-RAF1 Fusion; | | GIPC2-BRAF Fusion; |
| | | FGD5-RAF1 Fusion; | | GLI2-BRAF Fusion; JAK1- |
| | | GIPC2-BRAF Fusion; | | RAF1 Fusion; JHDM1D- |
| | | GLI2-BRAF Fusion; | | BRAF Fusion; KANK4- |
| | | JHDM1D-BRAF Fusion; | | RAF1 Fusion; LUC7L2- |
| | | LUC7L2-BRAF Fusion; | | BRAF Fusion; MBNL2- |
| | | MKRN1-BRAF Fusion; | | BRAF Fusion; MKRN1- |
| | | TMEM9-BRAF Fusion; | | BRAF Fusion; MYRIP- |
| | | RAF1 Rearrangement; | | BRAF Fusion; STRN3- |
| | | BRAF Rearrangement | | BRAF Fusion; SVOPL- |
| | | | | BRAF Fusion; TMEM9- |
| | | | | BRAF Fusion |
| Other or | 17/84 | T310I(2); D594G; | 21/81 | Amplification; D566E; |
| Multiple | (20.2%) | G469S; G596D; G596R; | (25.9%) | D594G; E26D; E501K; |
| Drivers | | N236K; S467L; T241P; | | G466V; G596R; K601N; |
| | | X327_splice; ARAF | | N236K; N581I & RAF1 |
| | | S214A; RAF1 S259Y; | | S257L; P403Lfs*8; Q94K; |
| | | T599K, V600 & | | R389H; S467L & deletion |
| | | Confounding Driver(3); | | exons 2-10; T241P; T310I; |
| | | N486_P490del & ROS1 | | truncation intron 8; V600E |
| | | Fusion | | & SND1-BRAF Fusion; |
| | | | | X327_splice; V600 & |
| | | | | Confounding Driver (2) |

*The Clinical Cohort includes a case series of 81 patients analyzed in this study from multiple institutions
**The Prevalence Cohort includes patients with molecular profiling data either from Perthera (a real-world database) or AACR GENIE (a public dataset, v6.0.1)

Case Series of BRAF-Mutated Pancreatic Cancer from KYT and Academic Collaborators

De-identified patient and genomic information was collected by collaborators from Dana-Farber, MD Anderson Cancer Center (MDACC), Memorial Sloan Kettering (MSK), PanCAN, Inova Schar Cancer Institute (ISCI) and Cedars-Sinai Medical Center (CSMC).

Individual patient charts were retrospectively reviewed, and clinical information was extracted.

PanCAN and Perthera initiated an IRB-approved observational registry trial to capture real-world outcomes across all lines of therapies and NGS testing results from CLIA-certified commercial laboratories in addition to proteomics/phosphoproteomics data as previously described [PMID: 32135080]. At MSK, DNA testing through MSK-IMPACT Assay (Memorial Sloan Kettering-Integrated Mutation Profiling of Actionable Cancer Targets) was used to identify the somatic genomic mutation profile of treated patients. BRAF somatic mutations in PC patients were identified using the cBioPortal. At MDACC samples were identified using the Molecular and Clinical Data Integration Platform (MOCLIP) of the Khalifa Institute for Personalized Cancer Therapy. Somatic DNA sequencing at Dana-Farber/Brigham and Women's Cancer Center was accomplished with an institutionally supported, CLIA-certified, hybrid-capture and massively parallel sequencing assay (OncoPanel) for adult and pediatric patients with cancer. Tumor biopsy samples from the ISCI and CSMC were sent to a CLIA-certified, CAP-accredited commercial laboratory (Foundation Medicine).

### Patient Outcomes Data

The investigators established the hypotheses and data points of interest after a series of scheduled meetings. Demographic, clinical descriptors, and outcomes data were collected under institution-specific IRB-approved protocols for each individual site. The data from each site was de-identified before being shared in a HIPAA-compliant database. A total of 81 patients with BRAF-mutated PC were identified from these institutions. however, population-level frequencies of BRAF alterations in PC could not be assessed since the denominator (i.e. the number of patients with adequate molecular profiling data) was not available outside the PanCAN/Perthera database. This motivated the need to combine public data with the PanCAN/Perthera dataset (as described above) to establish a Prevalence Cohort suitable for frequency analyses. Notably, this retrospective case series reflects a population of patients being treated predominantly at academic medical centers across coastal regions of the United States. Thus, this case series may not adequately represent important population-level factors (e.g. differences in insurance coverage, socioeconomic status, urban vs rural cohorts, and academic vs community settings) that can influence patient outcomes as well as access to targeted therapies either off label or on a clinical trial.

Demographic data, date of diagnosis, staging information, treatment history, and response to therapy were collected from all participating sites. Overall survival (OS) was calculated from the time of the patient's diagnosis of advanced PC (i.e. the date of recurrence following surgical resection or the date of initial diagnosis in the context of metastatic or unresectable disease) until death (survival event) or last follow-up (censored event). All OS survival analyses excluded patients who did not receive any therapy in the advanced setting. Progression-free survival (PFS) was calculated from the time of initiation of a therapy until discontinuation due to disease progression (survival event), cessation due to tolerability issues (censored event), or the last follow-up (censored event).

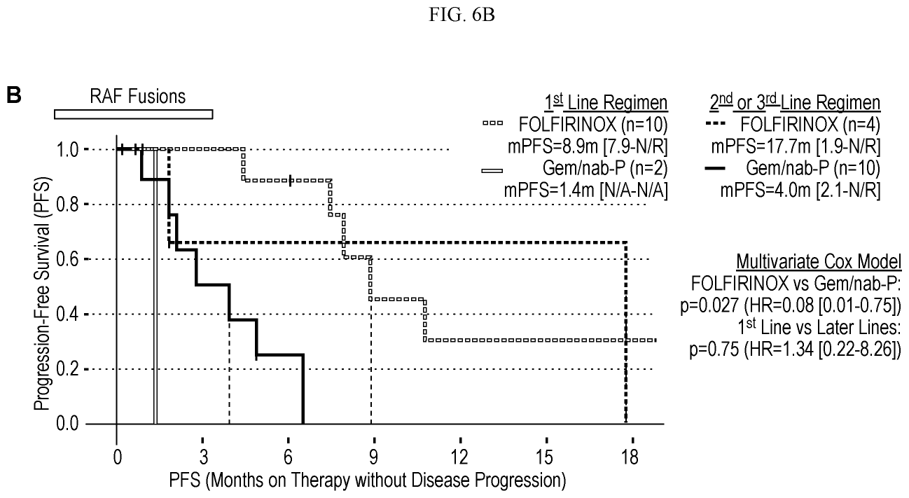

All survival analysis methods were implemented in an R/Bioconductor programming environment. All PFS analyses excluded patients with early stage or resectable disease. All OS analyses included only the subset of patients who were diagnosed with metastatic (Stage IV) disease at initial presentation. Patient outcomes related to PFS and OS were assessed using Cox proportional hazards regression models from the survival package and visualized using the survminer package. Multivariate Cox regression models were utilized to assess potentially confounding factors (e.g. 1^{st} line of therapy versus later lines of therapy for PFS analyeses; adenocarcinoma versus acinar cell carcinoma histology) when appropriate (see FIG. 6B). Differences in frequencies were assessed using Fisher's exact test.

### BRAF Alterations are Recurrent Events in Pancreatic Cancer

In a real-world cohort of 3781 PC patients with genomic testing results available, we identified 84 patients (2.2% of 3781 with PC) whose tumors harbored BRAF alterations within this “Prevalence Cohort” (Table 1).

We categorized each patient's molecular profile that included a BRAF alteration within the prevalence cohort into one of four subgroups: 26/84 (31%) Exon 15 (V600) mutations, 21/84 (25%) Exon 11 (non-V600) mutations/insertions/deletions, 20/84 (24%) oncogenic fusions (Fusions), or 17/84 (20%) non-canonical profiles (Other) with either multiple oncogenic drivers or atypical BRAF variants (Table 1). Although BRAF mutations are often mutually exclusive of mutations in other driver genes, we identified confounding genomic alterations (e.g. a KRAS/HRAS activating mutation or a ROS1 activating fusion event) within the molecular profiles of four patients with mutations that might otherwise have been assigned to the Exon 15 (10.3%, 3/29) or Exon 11 (4.5%, 1/22) subgroups. For those without any confounding drivers, the most common BRAF alterations included the canonical V600E mutation in Exon 15 (17/84, 20.2%), a recurring five-amino-acid in-frame deletion in the BRAF β3-αC loop within Exon 11 commonly referred to as ΔNVTAP or N486_P490del (16/84, 19.0%), and SND1-BRAF Fusions (9/84, 10.7%). These 3 specific BRAF alterations have distinct implications for targeted therapy (see Table 3) and form the basis of the Exon 15, Exon 11, and Fusion subgroups considered throughout this study.

Within the prevalence cohort, the proportion of BRAF alterations was higher (p=0.000000691, Fisher's Exact Test) in pancreatic acinar cell carcinoma (9/49, 18.4%) relative to pancreatic adenocarcinoma (64/3298, 1.9%). We observed that pancreatic acinar cell carcinoma frequently harbored BRAF fusion events (6/49, 12.2%) compared to mutation rates within pancreatic adenocarcinoma that are not expected to exceed 1% for any of the four BRAF mutational subgroups. BRAF Exon 15 (V600) mutations were also observed in rare PC histologies, with one in a pancreatoblastoma and in a solid pseudopapillary neoplasm.

### Patient Outcomes in BRAF-Altered Pancreatic Cancer

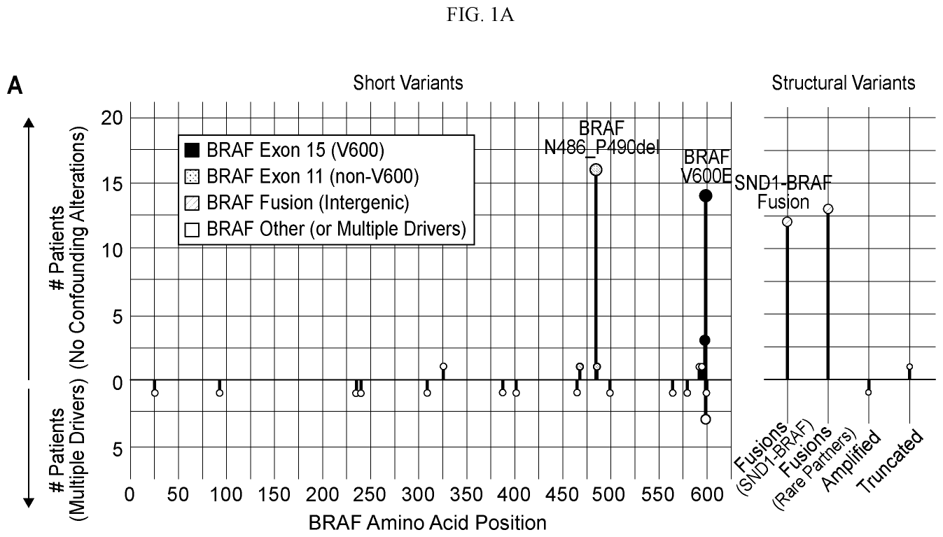

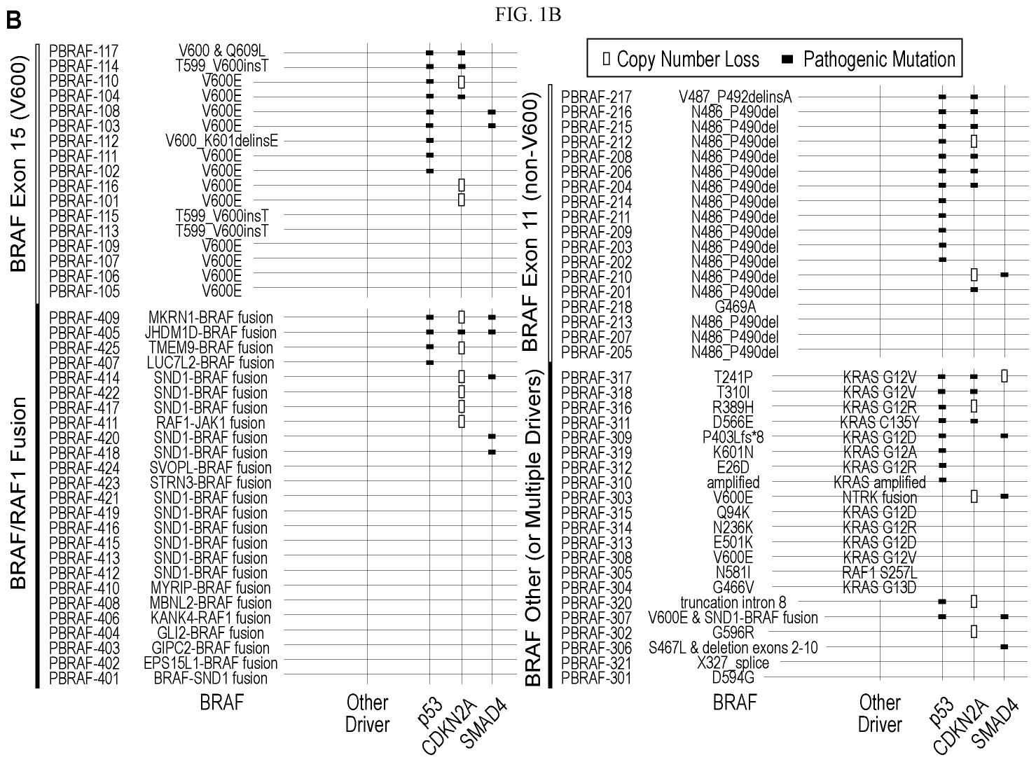

In a retrospective case series of PC patients, with clinically annotated outcomes data, we identified 81 patients with genomic alterations in BRAF (FIG. 1). In this cohort (referred to as the “Clinical Cohort” in Tables 1-2), the median age at diagnosis was 64 (42-86) and 40/81 were women (Table 2). The majority of patients presented with advanced disease 61/81 at initial diagnosis. The histologies were 62/81 adenocarcinoma, 14/81 acinar cell carcinoma or mixed acinar/neuroendocrine, 4/81 IPMN (excluded from the analysis cohort), and 1 pancreaticoblastoma (Table 2). The distribution of BRAF alterations (considered pathogenic by the genomic testing laboratory) were concentrated within Exons 11 and 15 (FIG. 1A), similar to the Prevalence Cohort (Table 1). Notably, 69 of the 81 tumor genomic profiles were KRAS WT. Additional genomic testing results were available for other commonly mutated genes in pancreatic cancer such as TP53 (43%), CDKN2A/B (36%), and SMAD4 (19%) (FIG. 1B). The median overall survival (mOS) of the analysis cohort (excludes IPMNs and cases with missing information) who presented with advanced disease (n=54) was 1.51y [95%0=1.11-2.01] with a median follow up of 1.22y and 38 total events. The median number of lines of therapy was 2.

TABLE 2
Summary of Patients with BRAF-mutated Pancreatic Cancer in the Clinical
Case Series Cohort and Overall Survival Analysis Cohorts

| | Clinical | | | | | | | |
|---|---|---|---|---|---|---|---|---|
| Baseline | Cohort | | OS Cohort | | OS Matched | | OS Unmatched | |
| Characteristic | (n = 81) | | (n = 52) | | (n = 16) | | (n = 36) | |
| Sex | | | | | | | | |
| Female | 40/81 | (49.4%) | 28/52 | (54%) | 11/16 | (69%) | 17/36 | (47%) |
| Male | 41/81 | (50.6%) | 24/52 | (46%) | 5/16 | (31%) | 19/36 | (53%) |
| Age at Diagnosis | | | | | | | | |
| >=64 | 41/81 | (50.6%) | 24/52 | (46%) | 5/16 | (31%) | 19/36 | (53%) |
| <64 | 40/81 | (49.4%) | 28/52 | (54%) | 11/16 | (69%) | 17/36 | (47%) |
| Pancreatic Subtype | | | | | | | | |
| Adenocarcinoma | 62/81 | (76%) | 43/52 | (83%) | 11/16 | (69%) | 32/36 | (89%) |
| Acinar Cell | 14/81 | (18%) | 9/52 | (17%) | 5/16 | (31%) | 4/36 | (11%) |
| Carcinoma | | | | | | | | |
| IPMN | 4/81 | (5%) | 0/52 | (0%) | 0/16 | (0%) | 0/36 | (0%) |
| Pancreatoblastoma | 1/81 | (1%) | 0/52 | (0%) | 0/16 | (0%) | 0/36 | (0%) |
| Stage at Diagnosis | | | | | | | | |
| 0/I (IPMN) | 4/81 | (5%) | 0/52 | (0%) | 0/16 | (0%) | 0/36 | (0%) |
| IIA/B | 15/81 | (19%) | 0/52 | (0%) | 0/16 | (0%) | 0/36 | (0%) |
| III | 5/81 | (6%) | 0/52 | (0%) | 0/16 | (0%) | 0/36 | (0%) |
| IV | 57/81 | (70%) | 52/52 | (100%) | 16/16 | (100%) | 36/36 | (100%) |
| Lines of Therapy | | | | | | | | |
| 3 lines or more | 24/81 | (30%) | 18/52 | (35%) | 10/16 | (62%) | 8/36 | (22%) |
| 2 lines | 17/81 | (21%) | 15/52 | (29%) | 1/16 | (6%) | 14/36 | (39%) |
| 1 line | 23/81 | (28%) | 19/52 | (37%) | 5/16 | (31%) | 14/36 | (39%) |
| None (Advanced) | 17/81 | (21%) | 0/52 | (0%) | 0/16 | (0%) | 0/36 | (0%) |

In this cohort, BRAF alterations occurring in adenocarcinoma subjects were evenly distributed between BRAF Exon 15 mutations (16/62), BRAF Exon 11 mutations (17/62), BRAF/RAF1 oncogenic fusion events (14/62) and the “Other” subgroup consisting of other genomic alterations, poorly-characterized variants, or genomic profiles with multiple drivers (14/62), In acinar cell tumors, the majority of alterations were fusion abnormalities (10/14) and Exon 15 mutations (V600) mutations (3/14). In the 4 subjects with IPMN, there was an Exon 15 V600 mutation, an Exon 11 in-frame deletion, and 2 missense mutations. One subject with a pancreaticoblastoma had a V600E alteration, which has not been reported previously.

### BRAF Alterations and Response to BRAF-Directed Therapy

In order to assess response to BRAF-directed therapy, genomic profiles from the clinical cohort were classified into one of four subgroupings (Table 1) as described above for the prevalence cohort: Exon 15 (V600) (17/81, 21.0%), Fusions (25/81, 30.9%), Exon 11 (non-V600) (18/81, 22.2%), or Other (21/81, 25.9%) which included BRAF mutations found alongside confounding oncogenic drivers or uncharacterized/atypical alterations that were not appropriate for assignment to the Exon 11, Exon 15, or Fusion subgroups (see Table 3 for additional details related to the rationale for each patient's subgrouping). Of the 17 BRAF^{V600E} alterations, 14 were mutually exclusive of additional alterations in BRAF, KRAS, or other potential driving alterations. If a known confounding driver was identified alongside any BRAF alteration (including 3 profiles with BRAF V600E mutations), they were grouped in Other. Activating KRAS mutations were mutually exclusive with BRAF fusion events (0/25 co-occurrences) within the clinical cohort (as well as the prevalence cohort); however, one tumor with a BRAF V600E mutation also had a SND1-BRAF fusion. The canonical BRAF ΔNVTAP mutation, BRAF N486_P490del, was the most common variant (n=16) within the Exon 11 cohort (n=18). This BRAF N486_P490del variant was mutually exclusive with KRAS mutations (n=0 overlap) in the clinical cohort (note: n=1 overlap with a ROS1 fusion from the prevalence cohort).

Our last category, termed “Other”, included 21 patients with BRAF variants that were interpreted as pathogenic by the genomic testing laboratory but not considered appropriate for the Exon 11, Exon 15, or Fusion subgroups when taking into consideration the entire tumor genomic profile. known to be RAS-dependent (e.g. BRAF “class 3” variants do not share the same implications for therapy as “class 1” V600 variants), found alongside a confounding driver mutation (e.g. KRAS), or not otherwise classifiable into the Exon 11, Exon 15, or Fusion categories (see Table 3 for patient-specific assignment details for this category). In this grouping, 15/21 had multiple distinct drivers. Of those with multiple drivers outside of the BRAF gene, 13/15 had KRAS alterations, one NTRK fusion, and a RAF1 mutation. Of the three patients with 2 alterations in the BRAF gene, one had both a V600E and a SND1-BRAF fusion, whereas another had both a BRAF S467L mutation with a complex BRAF Exon 2-10 deletion (unclear functional impact; however, similar alterations have been associated with acquired resistance to BRAF inhibitors, but no targeted therapies were documented in this patient's history). Each of the 17 non-canonical BRAF alterations were unique in this clinical cohort (FIG. 1). The only 2 protein-coding BRAF short variants identified in the absence of another driver are known to be RAS-dependent: D594G (kinase-dead variant impairs BRAF kinase activity but enhances heterodimer activity with wild type copies of RAF; vemurafenib-insensitive) and G596R (low kinase activity in vitro, vemurafenib-insensitive). Despite BRAF D594G and G596R being positioned within Exon 15 and their close proximity to the V600 hotspot region, we could not justify grouping these “Class 3” variants alongside mostly “Class 1” variants (high activity, RAS-independent, dimer-independent) within the Exon 15 subgroup when their functional consequences and downstream therapeutic implications are so drastically different.

TABLE 3

| | | | | | BRAF | | | BRAF | | | BRAF | Con- |
|---|---|---|---|---|---|---|---|---|---|---|---|---|
| | | BRAF | BRAF | BRAF | Dimer | Implications | | De- | BRAF | BRAF | Allele | found- |
| Patient | Cancer | Sub- | Sub- | Activity | Depen- | for BRAF | BRAF | scrip- | Refer- | Alter- | Fre- | ing |
| ID | Type | group | class | Type | dency | Inhibitors | Codon | tion | ences | ations | quency | Drivers |
| PBRAF- | Pancre- | Exon 15 | Class 1 | High | Mono- | Sensitive to | 600 | Mis- | PMID: | BRAF | 36.63% | |
| 101 | atic | (V600) | | Activity/ | mer- | canonical | | sense | 28783719 | V600E | | |
| | Adeno- | | | RAS- | specific | BRAF | | muta- | | | | |
| | carci- | | | Independent | | inhibitors | | tion | | | | |
| | noma | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Pancre- | Exon 15 | Class 1 | High | Mono- | Sensitive to | 600 | Mis- | PMID: | BRAF | N/A | |
| 102 | atic | (V600) | | Activity/ | mer- | canonical | | sense | 28783719 | V600E | | |
| | Adeno- | | | RAS- | specific | BRAF | | muta- | | | | |
| | carci- | | | Independent | | inhibitors | | tion | | | | |
| | noma | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Pancre- | Exon 15 | Class 1 | High | Mono- | Sensitive to | 600 | Mis- | PMID: | BRAF | N/A | |
| 103 | atic | (V600) | | Activity/ | mer- | canonical | | sense | 28783719 | V600E | | |
| | Adeno- | | | RAS- | specific | BRAF | | muta- | | | | |
| | carci- | | | Independent | | inhibitors | | tion | | | | |
| | noma | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Pancre- | Exon 15 | Class 1 | High | Mono- | Sensitive to | 600 | Mis- | PMID: | BRAF | N/A | |
| 104 | atic | (V600) | | Activity/ | mer- | canonical | | sense | 28783719 | V600E | | |
| | Adeno- | | | RAS- | specific | BRAF | | muta- | | | | |
| | carci- | | | Independent | | inhibitors | | tion | | | | |
| | noma | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Pancre- | Exon 15 | Class 1 | High | Mono- | Sensitive to | 600 | Mis- | PMID: | BRAF | N/A | |
| 105 | atic | (V600) | | Activity/ | mer- | canonical | | sense | 28783719 | V600E | | |
| | adeno- | | | RAS- | specific | BRAF | | muta- | | | | |
| | carci- | | | Independent | | inhibitors | | tion | | | | |
| | noma | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Acinar- | Exon 15 | Class 1 | High | Mono- | Sensitive to | 600 | Mis- | PMID: | BRAF | N/A | |
| 106 | Cell | (V600) | | Activity/ | mer- | canonical | | sense | 28783719 | V600E | | |
| | Carci- | | | RAS- | specific | BRAF | | muta- | | | | |
| | noma | | | Independent | | inhibitors | | tion | | | | |
| | | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Intra- | Exon 15 | Class 1 | High | Mono- | Sensitive to | 600 | Mis- | PMID: | BRAF | N/A | |
| 107 | ductal | (V600) | | Activity/ | mer- | canonical | | sense | 28783719 | V600E | | |
| | Pap- | | | RAS- | specific | BRAF | | muta- | | | | |
| | illary | | | Independent | | inhibitors | | tion | | | | |
| | Muci- | | | [Applicable | | (companion | | | | | | |
| | nous | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Pancre- | Exon 15 | Class 1 | High | Mono- | Sensitive to | 600 | Mis- | PMID: | BRAF | N/A | |
| 108 | atic | (V600) | | Activity/ | mer- | canonical | | sense | 28783719 | V600E | | |
| | Adeno- | | | RAS- | specific | BRAF | | muta- | | | | |
| | carci- | | | Independent | | inhibitors | | tion | | | | |
| | noma | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Pancre- | Exon 15 | Class 1 | High | Mono- | Sensitive to | 600 | Mis- | PMID: | BRAF | N/A | |
| 109 | atic | (V600) | | Activity/ | mer- | canonical | | sense | 28783719 | V600E | | |
| | Adeno- | | | RAS- | specific | BRAF | | muta- | | | | |
| | carci- | | | Independent | | inhibitors | | tion | | | | |
| | noma | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Pancre- | Exon 15 | Class 1 | High | Mono- | Sensitive to | 600 | Mis- | PMID: | BRAF | N/A | |
| 110 | atic | (V600) | | Activity/ | mer- | canonical | | sense | 28783719 | V600E | | |
| | Adeno- | | | RAS- | specific | BRAF | | muta- | | | | |
| | carci- | | | Independent | | inhibitors | | tion | | | | |
| | noma | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Acinar | Exon 15 | Class 1 | High | Mono- | Sensitive to | 600 | Mis- | PMID: | BRAF | 35.35% | |
| 111 | Cell | (V600) | | Activity/ | mer- | canonical | | sense | 28783719 | V600E | | |
| | Carci- | | | RAS- | specific | BRAF | | muta- | | | | |
| | noma | | | Independent | | inhibitors | | tion | | | | |
| | (Mixed | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Pancre- | Exon 15 | Uncon- | Unknown | Un- | Likely | 600 | In- | PMID: | BRAF | N/A | |
| 112 | atic | (V600) | firmed | | known | sensitive to | | frame | 24108467; | V600_ | | |
| | Adeno- | | (Class 1 | | | canonical | | inser- | PMID: | K601- | | |
| | carci- | | | | | BRAF | | tion/ | | delinsE | | |
| | noma | | | | | inhibitors | | dele- | | | | |
| | | | | | | (vemurafenib | | tion | | | | |
| | | | | | | sensitivity | | | | | | |
| | | | | | | confirmed | | | | | | |
| | | | | | | by case | | | | | | |
| | | | | | | reports | | | | | | |
| | | | | | | for a similar | | | | | | |
| PBRAF- | Pancre- | Exon 15 | Uncon- | Unknown | Un- | Likely | 599 | In- | PMID: | BRAF | N/A | |
| 113 | atic | (V600) | firmed | | known | sensitive to | | frame | 23833300; | T599_ | | |
| | Adeno- | | (Class 1 | | | canonical | | inser- | PMID: | V600- | | |
| | carci- | | | | | BRAF | | tion | | insT | | |
| | noma | | | | | inhibitors | | | | | | |
| | | | | | | (vemurafenib | | | | | | |
| | | | | | | sensitivity | | | | | | |
| | | | | | | confirmed | | | | | | |
| | | | | | | by case | | | | | | |
| | | | | | | reports | | | | | | |
| | | | | | | for this | | | | | | |
| PBRAF- | Pancre- | Exon 15 | Uncon- | Unknown | Un- | Likely | 599 | In- | PMID: | BRAF | 6.90% | |
| 114 | atic | (V600) | firmed | | known | sensitive to | | frame | 23833300; | T599_ | | |
| | Adeno- | | (Class 1 | | | canonical | | inser- | PMID: | V600 | | |
| | carci- | | | | | BRAF | | tion | | insT | | |
| | noma | | | | | inhibitors | | | | | | |
| | | | | | | (vemurafenib | | | | | | |
| | | | | | | sensitivity | | | | | | |
| | | | | | | confirmed | | | | | | |
| | | | | | | by case | | | | | | |
| | | | | | | reports | | | | | | |
| | | | | | | for this | | | | | | |
| PBRAF- | Pancre- | Exon 15 | Uncon- | Unknown | Un- | Likely | 599 | In- | PMID: | BRAF | N/A | |
| 115 | atic | (V600) | firmed | | known | sensitive to | | frame | 23833300; | T599_ | | |
| | Adeno- | | (Class 1 | | | canonical | | inser- | PMID: | V600 | | |
| | carci- | | | | | BRAF | | tion | | insT | | |
| | noma | | | | | inhibitors | | | | | | |
| | | | | | | (vemurafenib | | | | | | |
| | | | | | | sensitivity | | | | | | |
| | | | | | | confirmed | | | | | | |
| | | | | | | by case | | | | | | |
| | | | | | | reports | | | | | | |
| | | | | | | for this | | | | | | |
| PBRAF- | Acinar | Exon 15 | Class 1 | High | Mono- | Sensitive to | 600 | Mis- | PMID: | BRAF | 36.21% | |
| 116 | Cell | (V600) | | Activity/ | mer- | canonical | | sense | 28783719 | V600E | | |
| | Carci- | | | RAS- | specific | BRAF | | muta- | | | | |
| | noma | | | Independent | | inhibitors | | tion | | | | |
| | (Mixed | | | [Applicable | | (companion | | | | | | |
| | | | | to | | diagnostic | | | | | | |
| | | | | both BRAF | | marker for | | | | | | |
| | | | | alterations] | | vemurafenib/ | | | | | | |
| | | | | | | dabrafenib/ | | | | | | |
| | | | | | | encorafenib) | | | | | | |
| PBRAF- | Pancre- | Exon 15 | Class 1 | High | Mono- | Likely | 600 | Mis- | PMID: | BRAF | N/A | [insuf- |
| 117 | atic | (V600) | & | Activity/ | mer- | sensitive to | | sense | 28783719 | V600E | | ficient |
| | Adeno- | | Unknow | RAS- | specific | canonical | | muta- | | & | | evi- |
| | carci- | | | Independent | [NOTE: | BRAF | | tions | | BRAF | | dence |
| | noma | | | [Applicable | | inhibitors | | | | | | avail- |
| | | | | to | | (companion | | | | | | able |
| | | | | both BRAF | | diagnostic | | | | | | to |
| | | | | alterations] | | marker for | | | | | | |
| PBRAF- | Pancre- | Exon 11 | Uncon- | High | Mono- | Experi- | 486 | In- | PMID: | BRAF | 26.46% | |
| 201 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | N/A | |
| 202 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | N/A | |
| 203 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | N/A | |
| 204 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | N/A | |
| 205 | atic | (non- | firmed | Activity/ | mer- | mentally | | frame | 26996308 | N486_ | | |
| | Adeno; | | | RAS- | specific | vemurafenib- | | dele- | | P49 | | |
| | carci- | | | Independent | | insensitive | | tion | | | | |
| | noma; | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | N/A | |
| 206 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | 15.42% | |
| 207 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | 26.62% | |
| 208 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | 9.73% | |
| 209 | atic | (non- | firmed | Activity/ | mer- | mentally | | frame | 26996308 | N486_ | | |
| | Adeno- | V600) | (Class 1 | RAS- | specific | vemurafenib- | | dele- | | P490- | | |
| | carci- | | | Independent | | insensitive | | tion | | del | | |
| | noma | | | | | BUT | | (delta- | | | | |
| | | | | | | dabrafenib- | | NVTA | | | | |
| | | | | | | sensitive; | | | | | | |
| | | | | | | potentially | | | | | | |
| | | | | | | sensitive | | | | | | |
| | | | | | | to pan-RAF | | | | | | |
| | | | | | | inhibitors | | | | | | |
| PBRAF- | Pancre- | Exon 11 | Uncon- | High | Mono- | Experi- | 486 | In- | PMID: | BRAF | N/A | |
| 210 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | N/A | |
| 211 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | N/A | |
| 212 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Intra | Exon 11 | Uncon- | High | Mono- | Experi- | 486 | In- | PMID: | BRAF | N/A | |
| 213 | ductal- | (non- | firmed | Activity/ | mer- | mentally | | frame | 26996308 | N486_ | | |
| | Pap- | V600) | (Class 1 | RAS- | specific | vemurafenib- | | dele- | | P49 | | |
| | illary | | | Independent | | insensitive | | tion | | Odel | | |
| | Muci- | | | | | BUT | | (delta- | | | | |
| | nous | | | | | dabrafenib- | | NVTA | | | | |
| | | | | | | sensitive; | | | | | | |
| | | | | | | potentially | | | | | | |
| | | | | | | sensitive | | | | | | |
| | | | | | | to pan-RAF | | | | | | |
| | | | | | | inhibitors | | | | | | |
| PBRAF- | Pancre- | Exon 11 | Uncon- | High | Mono- | Experi- | 486 | In- | PMID: | BRAF | N/A | |
| 214 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | N/A | |
| 215 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Uncon- | High | Mono- | Experi- | 486 | In- | PMID: | BRAF | N/A | |
| 216 | atic | (non- | firmed | Activity/ | mer- | mentally | | frame | 26996308 | N486_ | | |
| | Adeno- | | | RAS- | specific | vemurafenib- | | dele- | | P49 | | |
| | carci- | | | Independent | | insensitive | | tion | | | | |
| | noma | | | | | BUT | | | | | | |
| | | | | | | dabrafenib- | | | | | | |
| | | | | | | sensitive; | | | | | | |
| | | | | | | potentially | | | | | | |
| PBRAF- | Pancre- | Exon 11 | Un- | Unknown | Un- | Presumably | 487 | In- | PMID: | BRAF | 6.33% | |
| 217 | atic | (non- | known | | known | vemurafenib- | | frame | 26996308 | V487_ | | |
| | Adeno- | V600) | | | | insensilive | | inser- | | P492- | | |
| | carci- | | | | | due to | | tion/ | | delinsA | | |
| | noma | | | | | similarity | | dele- | | | | |
| | | | | | | to the | | tion | | | | |
| | | | | | | NVTAP | | | | | | |
| | | | | | | deletion; | | | | | | |
| | | | | | | potentially | | | | | | |
| | | | | | | sensitive | | | | | | |
| | | | | | | to pan-RAF | | | | | | |
| PBRAF- | Pancre- | Exon 11 | Class 2 | High | Dimer- | Experi- | 469 | Mis- | PMID: | BRAF | 3.12% | |
| 218 | atic | (non- | | Activity/ | depen- | mentally | | sense | 28783719 | G469A | | |
| | Adeno- | | | RAS- | dent | vemurafenib- | | muta- | | | | |
| | carci- | | | Independent | | insensilive; | | tion | | | | |
| | noma | | | | | potentially | | | | | | |
| | | | | | | sensitive | | | | | | |
| | | | | | | to pan-RAF | | | | | | |
| PBRAF- | Pancre- | Other or | Class 3 | No | Dimer- | Experi- | 594 | Mis- | PMID: | BRAF | 26.73% | |
| 301 | atic | Multiple | | Activity/ | depen- | mentally | | sense | 28783719 | D594G | | |
| | Adeno- | | | RAS- | dent | vemurafenib- | | muta- | | | | |
| | carci- | | | Dependent | | insensilive; | | tion | | | | |
| | noma | | | | | unlikely | | | | | | |
| | | | | | | to confer | | | | | | |
| | | | | | | sensitivity | | | | | | |
| | | | | | | to other | | | | | | |
| PBRAF- | Pancre- | Other or | Class 3 | Low | Dimer- | Unlikely | 596 | Mis- | PMID: | BRAF | N/A | |
| 302 | atic | Multiple | | Activity/ | depen- | to confer | | sense | 28783719 | G596R | | |
| | Adeno- | | | RAS- | dent | sensitivity | | muta- | | | | |
| | carci- | | | Dependent | | to either | | tion | | | | |
| | noma | | | | | canonical | | | | | | |
| | | | | | | BRAF | | | | | | |
| | | | | | | inhibitors | | | | | | |
| | | | | | | or pan-RAF | | | | | | |
| PBRAF- | Pancre- | Other or | Class 1 | High | Mono- | Sensitive | 600 | Mis- | PMID: | BRAF | N/A | NTRK |
| 303 | atic | Multiple | | Activity/ | mer- | to canonical | | sense | 28783719 | V600E | | fusion |
| | Adeno- | Drivers | | RAS- | specific | BRAF | | muta- | | | | |
| | carci- | | | Independent | | inhibitors | | tion | | | | |
| | noma | | | [Applicable | | (companion | | | | | | |
| | | | | to | | diagnostic | | | | | | |
| | | | | both | | marker for | | | | | | |
| | | | | BRAF | | vemurafenib/ | | | | | | |
| | | | | alterations] | | dabrafenib/ | | | | | | |
| | | | | | | encorafenib) | | | | | | |
| PBRAF- | Intra- | Other or | Class 3 | Low | Dimer- | Experi- | 466 | Mis- | PMID: | BRAF | N/A | KRAS |
| 304 | ductal | Multiple | | Activity/ | depen- | mentally | | sense | 28783719 | G466V | | G13D |
| | Pap- | Drivers | | RAS- | dent | vemurafenib- | | muta- | | | | |
| | illary | | | Dependent | | insensilive; | | tion | | | | |
| | Muci- | | | | | unlikely | | | | | | |
| | nous | | | | | to confer | | | | | | |
| | | | | | | sensitivity | | | | | | |
| | | | | | | to other | | | | | | |
| | | | | | | BRAF | | | | | | |
| | | | | | | or pan-RAF | | | | | | |
| | | | | | | inhibitors | | | | | | |
| PBRAF- | Am- | Other or | Class 3 | Low | Dimer- | Experi- | 581 | Mis- | PMID: | BRAF | N/A | Multiple |
| 305 | pullary | Multiple | | Activity/ | depen- | mentally | | sense | 28783719 | N581I& | | RAF |
| | Adeno- | | | RAS- | dent | vemurafenib- | | muta- | | | | Alter- |
| | carci- | | | Dependent | | insensitive; | | tion | | | | ations |
| | noma | | | | | unlikely | | | | | | |
| | | | | | | to confer | | | | | | |
| | | | | | | sensitivity | | | | | | |
| | | | | | | to other | | | | | | |
| PBRAF- | Pancre- | Other or | Class 3 | Low | Dimer- | Experi- | 467 | Other | PMID: | BRAF | N/A | Multiple |
| 306 | atic | Multiple | & | Activity/ | depen- | mentally | | (Intra- | 28783719 | S467L & | | RAF |
| | Adeno- | Drivers | Unknow | RAS- | dent | vemurafenib- | | genic | | BRAF | | Alter- |
| | carci- | | | Dependent | | insensitive | | Re- | | | | ations |
| | noma | | | | | (S467L; | | arrange- | | | | |
| | | | | | | unknown for | | m | | | | |
| | | | | | | exonal | | | | | | |
| | | | | | | deletion); | | | | | | |
| | | | | | | unlikely | | | | | | |
| | | | | | | to confer | | | | | | |
| | | | | | | sensitivity | | | | | | |
| | | | | | | to other | | | | | | |
| PBRAF- | Pancre- | Other or | Class 1 | High | Mono- | Conflicting | 600 | Mis- | PMID: | BRAF | N/A | Multiple |
| 307 | atic | Multiple | & Class | Activity/ | mer- | markers of | | sense | 28783719 | V600E& | | RAF |
| | Adeno- | Drivers | 2 | RAS- | specific | sensitivty to | | muta- | | SND1- | | Alter- |
| | carci- | | | Independent | [NOTE: | canonical | | tion & | | | | ations |
| | noma | | | [Applicable | | BRAF | | inter- | | | | |
| | | | | to | | inhibitors | | genic | | | | |
| | | | | both BRAF | | (fusions are | | | | | | |
| | | | | alterations] | | associated | | | | | | |
| | | | | | | with | | | | | | |
| | | | | | | vemurafenib- | | | | | | |
| PBRAF- | Pancre- | Other or | Class 1 | High | Mono- | Implications | 600 | Mis- | PMID: | BRAF | N/A | KRAS |
| 308 | atic | Multiple | | Activity/ | mer- | for canonical | | sense | 28783719 | V600E | | G12V |
| | Adeno- | | | RAS- | specific | BRAF | | muta- | | | | |
| | carci- | | | Independent | | inhibitors are | | tion | | | | |
| | noma | | | [Applicable | | confounded | | | | | | |
| | | | | to | | by the | | | | | | |
| | | | | both BRAF | | | | | | | | |
| | | | | alterations] | | | | | | | | |
| PBRAF- | Pancre- | Other or | Unknown | Unknown | Un- | Unlikely | 403 | Frame- | | BRAF | N/A | KRAS |
| 309 | atic | Multiple | | | known | to confer | | shift | | P403Lfs* | | G12D |
| | Adeno- | | | | | sensitivity | | muta- | | 8 | | |
| | carci- | | | | | to either | | tion | | | | |
| | noma | | | | | canonical | | | | | | |
| | | | | | | BRAF | | | | | | |
| | | | | | | inhibitors or | | | | | | |
| | | | | | | pan-RAF | | | | | | |
| PBRAF- | Pancre- | Other or | Unknown | Unknown | Un- | Likely | CNV | Copy | | BRAF | N/A | KRAS |
| 310 | atic | Multiple | | | known | insensitive | | number | | amplified | | Ampli- |
| | Adeno- | Drivers | | | | to canonical | | alter- | | | | fication |
| | carci- | | | | | BRAF | | ation | | | | |
| | noma | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Pancre- | Other or | Unknown | Unknown | Un- | Unlikely | 566 | Mis- | | BRAF | N/A | KRAS |
| 311 | atic | Multiple | | | known | to confer | | sense | | D566E | | C135Y |
| | Adeno- | Drivers | | | | sensitivity | | muta- | | | | |
| | carci- | | | | | to either | | tion | | | | |
| | noma | | | | | canonical | | | | | | |
| | | | | | | BRAF | | | | | | |
| | | | | | | inhibitors or | | | | | | |
| | | | | | | pan-RAF | | | | | | |
| | | | | | | inhibitors | | | | | | |
| PBRAF- | Pancre- | Other or | Unknown | Unknown | Un- | Unlikely | 26 | Mis- | | BRAF | N/A | KRAS |
| 312 | atic | Multiple | | | known | to confer | | sense | | E26D | | G12R |
| | Adeno- | | | | | sensitivity | | muta- | | | | |
| | carci- | | | | | to either | | tion | | | | |
| | noma | | | | | canonical | | | | | | |
| | | | | | | BRAF | | | | | | |
| | | | | | | inhibitors or | | | | | | |
| | | | | | | pan-RAF | | | | | | |
| | | | | | | inhibitors | | | | | | |
| PBRAF- | Pancre- | Other or | Unknown | Unknown | Un- | Unlikely | 501 | Mis- | | BRAF | N/A | KRAS |
| 313 | atic | Multiple | | | known | to confer | | sense | | E501K | | G12D |
| | Adeno- | Drivers | | | | sensitivity | | muta- | | | | |
| | carci- | | | | | to either | | tion | | | | |
| | noma | | | | | canonical | | | | | | |
| | | | | | | BRAF | | | | | | |
| | | | | | | inhibitors or | | | | | | |
| | | | | | | pan-RAF | | | | | | |
| | | | | | | inhibitors | | | | | | |
| PBRAF- | Intra- | Other or | Unknown | Unknown | Un- | Unlikely | 236 | Mis- | | BRAF | N/A | KRAS |
| 314 | ductal | Multiple | | | known | to confer | | sense | | N236K | | G12R |
| | Pap- | Drivers | | | | sensitivity | | muta- | | | | |
| | illary | | | | | to either | | tion | | | | |
| | Muci- | | | | | canonical | | | | | | |
| | nous | | | | | BRAF | | | | | | |
| | | | | | | inhibitors or | | | | | | |
| | | | | | | pan-RAF | | | | | | |
| | | | | | | inhibitors | | | | | | |
| PBRAF- | Pancre- | Other or | Unknown | Unknown | Un- | Unlikely | 94 | Mis- | | BRAF | N/A | KRAS |
| 315 | atic | Multiple | | | known | to confer | | sense | | 094K | | G12D |
| | Adeno- | | | | | sensitivity | | muta- | | | | |
| | carci- | | | | | to either | | tion | | | | |
| | noma | | | | | canonical | | | | | | |
| | | | | | | BRAF | | | | | | |
| | | | | | | inhibitors or | | | | | | |
| | | | | | | pan-RAF | | | | | | |
| PBRAF- | Pancre- | Other or | Unknown | Unknown | Un- | Unlikely | 389 | Mis- | | BRAF | N/A | KRAS |
| 316 | atic | Multiple | | | known | to confer | | sense | | R389H | | G12R |
| | Adeno- | | | | | sensitivity | | muta- | | | | |
| | carci- | | | | | to either | | tion | | | | |
| | noma | | | | | canonical | | | | | | |
| | | | | | | BRAF | | | | | | |
| | | | | | | inhibitors or | | | | | | |
| | | | | | | pan-RAF | | | | | | |
| PBRAF- | Pancre- | Other or | Unknown | Unknown | Un- | Unlikely | 241 | Mis- | | BRAF | N/A | KRAS |
| 317 | atic | Multiple | | | known | to confer | | sense | | T241P | | G12V |
| | Adeno- | | | | | sensitivity | | muta- | | | | |
| | carci- | | | | | to either | | tion | | | | |
| | noma | | | | | canonical | | | | | | |
| | | | | | | BRAF | | | | | | |
| | | | | | | inhibitors or | | | | | | |
| | | | | | | pan-RAF | | | | | | |
| PBRAF- | Pancre- | Other or | Unknown | Unknown | Un- | Unlikely | 310 | Mis- | | BRAF | N/A | KRAS |
| 318 | atic | Multiple | | | known | to confer | | sense | | T310I | | G12V |
| | Adeno- | | | | | sensitivity | | muta- | | | | |
| | carci- | | | | | to either | | tion | | | | |
| | noma | | | | | canonical | | | | | | |
| | | | | | | BRAF | | | | | | |
| | | | | | | inhibitors or | | | | | | |
| | | | | | | pan-RAF | | | | | | |
| PBRAF- | Pancre- | Other or | Class 2 | High | Dimer- | Likely | 601 | Mis- | PMID: | BRAF | N/A | KRAS |
| 319 | atic | Multiple | | Activity/ | depen- | insensitive | | sense | 28783719 | K601N | | G12A |
| | Adeno- | Drivers | | RAS- | dent | to canonical | | muta- | | | | |
| | carci- | | | Independent | | BRAF | | tion | | | | |
| | noma | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Pancre- | Other or | Unknown | Unknown | Un- | Likely | FUS | Intra- | | BRAF | N/A | |
| 320 | atic | Multiple | | | known | insensitive | | genic | | trun- | | |
| | Adeno- | Drivers | | | | to canonical | | struc- | | cation | | |
| | carci- | | | | | BRAF | | tural | | intron 8 | | |
| | noma | | | | | inhibitors | | variant | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Acinar | Other or | Unknown | Unknown | Un- | Unlikely | 327 | Splice | | BRAF | N/A | |
| 321 | Cell | Multiple | | | known | to confer | | site | | X327_ | | |
| | Carci- | | | | | sensitivity | | muta- | | splic | | |
| | noma | | | | | to either | | tion | | | | |
| | | | | | | canonical | | | | | | |
| | | | | | | BRAF | | | | | | |
| | | | | | | inhibitors or | | | | | | |
| | | | | | | pan-RAF | | | | | | |
| PBRAF- | Pancre- | Class 2 | Class 2 | High | Dimer- | Likely | FUS | Inter- | PMID: | SND1- | N/A | |
| 401 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Pancre- | Class 2 | Class 2 | High | Dimer- | Likely | FUS | Inter- | PMID: | EPS15L1- | N/A | |
| 402 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Acinar | Class 2 | Class 2 | High | Dimer- | Likely | FUS | Inter- | PMID: | GIPC2- | N/A | |
| 403 | Cell | (Fusion) | | Activity/ | depen- | insensitive | | genic | 28783719 | BRAF | | |
| | Carci- | | | RAS- | dent | to canonical | | fusion | | fusion | | |
| | noma | | | Independent | | BRAF | | event | | | | |
| | (Mixed | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Acinar | Class 2 | Class 2 | High | Dimer- | Likely | FUS | Inter- | PMID: | GLI2- | N/A | |
| 404 | Cell | (Fusion) | | Activity/ | depen- | insensitive | | genic | 28783719 | BRAF | | |
| | Carci- | | | RAS- | dent | to canonical | | fusion | | fusion | | |
| | noma | | | Independent | | BRAF | | event | | | | |
| | | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Pancre- | Class 2 | Class 2 | High | Dimer- | Likely | FUS | Inter- | PMID: | JHDM1D- | N/A | |
| 405 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Acinar | Class 2 | Class 2 | High | Dimer- | Likely | FUS | Inter- | PMID: | KANK4- | N/A | |
| 406 | Cell | (Fusion) | | Activity/ | depen- | insensitive | | genic | 28783719 | RAF1 | | |
| | Carci- | | | RAS- | dent | to canonical | | fusion | | fusion | | |
| | noma | | | Independent | | BRAF | | event | | | | |
| | | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Pancre- | Class 2 | Class 2 | High | Dimer- | Likely | FUS | Inter- | PMID: | LUC7L2- | N/A | |
| 407 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Pancre- | Class 2 | Class 2 | High | Dimer- | Likely | FUS | Inter- | PMID: | MBNL2- | N/A | |
| 408 | atic | (Fusion) | | Activity/ | depen- | insensitive | | genic | 28783719 | BRAF | | |
| | Adeno; | | | RAS- | dent | to canonical | | fusion | | fusion | | |
| | carci- | | | Independent | | BRAF | | event | | | | |
| | noma; | | | [Un- | | inhibitors | | | | | | |
| | Poorly | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Pancre- | Class 2 | Class 2 | High | Dimer- | Likely | FUS | Inter- | PMID: | MKRN1- | N/A | |
| 409 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Pancre- | Fusion | Class 2 | High | Dimer- | Likely | FUS | Inter- | PMID: | MYRIP- | N/A | |
| 410 | atic | | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Pancre- | Class 2 | Class 2 | High | Dimer- | Likely | FUS | Inter- | PMID: | RAF1- | N/A | |
| 411 | atic | (Fusion) | | Activity/ | depen- | insensitive | | genic | 28783719 | JAK1 | | |
| | Adeno- | | | RAS- | dent | to canonical | | fusion | | fusion | | |
| | carci- | | | Independent | | BRAF | | event | | | | |
| | noma | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Pancre- | Class 2 | Class 2 | High | Dimer- | Likely | FUS | Inter- | PMID: | SND1- | N/A | |
| 412 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Acinar | Class 2 | Class 2 | High | Dimer- | Likely | FUS | Inter- | PMID: | SND1- | N/A | |
| 413 | Cell | (Fusion) | | Activity/ | depen- | insensitive | | genic | 28783719 | BRAF | | |
| | Carci- | | | RAS- | dent | to canonical | | fusion | | fusion | | |
| | noma | | | Independent | | BRAF | | event | | | | |
| | | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Pancre- | Class 2 | Class 2 | High | Dimer- | Likely | FUS | Inter- | PMID: | SND1- | N/A | |
| 414 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Pancre- | Class 2 | Class 2 | High | Dimer- | Likely | FUS | Inter- | PMID: | SND1- | N/A | |
| 415 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Acinar | Class 2 | Class 2 | High | Dimer- | Likely | FUS | Inter- | PMID: | SND1- | N/A | |
| 416 | Cell | (Fusion) | | Activity/ | depen- | insensitive | | genic | 28783719 | BRAF | | |
| | Carci- | | | RAS- | dent | to canonical | | fusion | | fusion | | |
| | noma | | | Independent | | BRAF | | event | | | | |
| | (Mixed | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Acinar | Class 2 | Class 2 | High | Dimer- | Likely | FUS | Inter- | PMID: | SND1- | N/A | |
| 417 | Cell | (Fusion) | | Activity/ | depen- | insensitive | | genic | 28783719 | BRAF | | |
| | Carci- | | | RAS- | dent | to canonical | | fusion | | fusion | | |
| | noma | | | Independent | | BRAF | | event | | | | |
| | | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Pancre- | Class 2 | Class 2 | High | Dimer- | Likely | FUS | Inter- | PMID: | SND1- | N/A | |
| 418 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Acinar | Class 2 | Class 2 | High | Dimer- | Likely | FUS | Inter- | PMID: | SND1- | N/A | |
| 419 | Cell | (Fusion) | | Activity/ | depen- | insensitive | | genic | 28783719 | BRAF | | |
| | Carci- | | | RAS- | dent | to canonical | | fusion | | fusion | | |
| | noma | | | Independent | | BRAF | | event | | | | |
| | (Mixed | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Acinar | Class 2 | Class 2 | High | Dimer- | Likely | FUS | Inter- | PMID: | SND1- | N/A | |
| 420 | Cell | (Fusion) | | Activity/ | depen- | insensitive | | genic | 28783719 | BRAF | | |
| | Carci- | | | RAS- | dent | to canonical | | fusion | | fusion | | |
| | noma | | | Independent | | BRAF | | event | | | | |
| | (Mixed | | | | | inhibitors | | | | | | |
| | Ductal) | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Acinar | Class 2 | Class 2 | High | Dimer- | Likely | FUS | Inter- | PMID: | SND1- | N/A | |
| 421 | Cell | (Fusion) | | Activity/ | depen- | insensitive | | genic | 28783719 | BRAF | | |
| | Carci- | | | RAS- | dent | to canonical | | fusion | | fusion | | |
| | noma | | | Independent | | BRAF | | event | | | | |
| | (Mixed | | | | | inhibitors | | | | | | |
| | | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Acinar | Class 2 | Class 2 | High | Dimer- | Likely | FUS | Inter- | PMID: | SND1- | N/A | |
| 422 | Cell | (Fusion) | | Activity/ | depen- | insensitive | | genic | 28783719 | BRAF | | |
| | Carci- | | | RAS- | dent | to canonical | | fusion | | fusion | | |
| | noma | | | Independent | | BRAF | | event | | | | |
| | (Mixed | | | | | inhibitors | | | | | | |
| | Ductal) | | | | | (e.g. | | | | | | |
| | | | | | | vemurafenib/ | | | | | | |
| | | | | | | dabrafenib/ | | | | | | |
| | | | | | | encorafenib); | | | | | | |
| PBRAF- | Pancre- | Class 2 | Class 2 | High | Dimer- | Likely | FUS | Inter- | PMID: | STRN3- | N/A | |
| 423 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Pancre- | Class 2 | Class 2 | High | Dimer- | Likely | FUS | Inter- | PMID: | SVOPL- | N/A | |
| 424 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| PBRAF- | Pancre- | Class 2 | Class 2 | High | Dimer- | Likely | FUS | Inter- | PMID: | TMEM9- | N/A | |
| 425 | atic | (Fusion) | | Activity/ | depen- | insensitive | | genic | 28783719 | BRAF | | |
| | Adeno- | | | RAS- | dent | to canonical | | fusion | | fusion | | |
| | carci- | | | Independent | | BRAF | | event | | | | |
| | noma | | | [Un- | | inhibitors | | | | | | |
| | | | | confirmed | | (e.g. | | | | | | |
| | | | | for this | | vemurafenib/ | | | | | | |
| | | | | specific | | dabrafenib/ | | | | | | |
| | | | | fusion | | encorafenib); | | | | | | |
| | | | | event] | | | | | | | | |
| | | | | | | | | | | | OS | |
| | | | | | | | | | | | (Ratio- | |
| | | | | | | | | | | | nale | |
| | KRAS | p53 | CDKN2A | | | | | OS | OS | OS | for | Target- |
| Patient | Alter- | Alter- | Alter- | SMAD4 | Age | | Stage | (Event | (Event | (Analysis | Exclu- | ed |
| ID | ations | ations | ations | Alterations | at Dx | Sex | at Dx | Days) | Status) | Cohort) | sion) | Therapy |
| PBRAF- | | | CDKN2A/ | | 53 | F | IV | 652 | Deceased | Included | | un- |
| 101 | | | B loss | | | | | | | | | matched |
| PBRAF- | | G245D | | | 83 | F | IV | 141 | Deceased | Excluded | No | other |
| 102 | | | | | | | | | | | treat- | |
| | | | | | | | | | | | ment | |
| | | | | | | | | | | | docu- | |
| | | | | | | | | | | | mented | |
| | | | | | | | | | | | in the | |
| | | | | | | | | | | | ad- | |
| | | | | | | | | | | | vanced | |
| PBRAF- | | G266R | | SMAD4 | 63 | F | III | 408 | Followup | Excluded | Initally | un- |
| 103 | | | | R445* | | | | | | | pre- | matched |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | C124L | M52* | | 66 | M | IV | 168 | Followup | Included | | un- |
| 104 | | | | | | | | | | | | matched |
| PBRAF- | | | | | 72 | M | IIA | 237 | Followup | Excluded | Initally | other |
| 105 | | | | | | | | | | | pre- | |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | | | | 65 | F | IV | 1688 | Followup | Included | | matched |
| 106 | | | | | | | | | | | | |
| PBRAF- | | | | | 74 | F | I | 677 | Followup | Excluded | Initally | other |
| 107 | | | | | | | | | | | pre- | |
| | | | | | | | | | | | sented | |
| | | | | | | | | | | | with | |
| | | | | | | | | | | | early | |
| | | | | | | | | | | | stage | |
| PBRAF- | | R209Kfs* | | SMAD4 | 65 | F | IV | 46 | Deceased | Excluded | No | other |
| 108 | | 6 | | D537H | | | | | | | treat- | |
| | | | | | | | | | | | ment | |
| | | | | | | | | | | | docu- | |
| | | | | | | | | | | | mented | |
| | | | | | | | | | | | in the | |
| | | | | | | | | | | | ad- | |
| | | | | | | | | | | | vanced | |
| PBRAF- | | | | | 55 | F | IV | 1492 | Deceased | Included | | un- |
| 109 | | | | | | | | | | | | matched |
| PBRAF- | | A76fs*73 | CDKN2A/ | | 43 | M | IV | 460 | Deceased | Included | | un- |
| 110 | | | B loss | | | | | | | | | matched |
| PBRAF- | | S183* | | | 51 | F | IIB | 363 | Followup | Excluded | Initally | other |
| 111 | | | | | | | | | | | pre- | |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | P153fs* | | | 70 | M | IV | 202 | Deceased | Included | | un- |
| 112 | | 28 | | | | | | | | | | matched |
| PBRAF- | | | | | 84 | F | II | 809 | Followup | Excluded | Initally | other |
| 113 | | | | | | | | | | | pre- | |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | R342* | R24fs*20 | | 63 | M | IV | 49 | Followup | Excluded | No | other |
| 114 | | | | | | | | | | | treat- | |
| | | | | | | | | | | | ment | |
| | | | | | | | | | | | docu- | |
| | | | | | | | | | | | mented | |
| | | | | | | | | | | | in the | |
| | | | | | | | | | | | ad- | |
| | | | | | | | | | | | vanced | |
| PBRAF- | | | | | 64 | M | IV | 702 | Deceased | Included | | matched |
| 115 | | | | | | | | | | | | |
| PBRAF- | | | CDKN2A | | 62 | F | IV | 815 | Deceased | Included | | matched |
| 116 | | | loss | | | | | | | | | |
| PBRAF- | | Y220C | A20fs; | | 48 | F | IV | 133 | Followup | Included | | un- |
| 117 | | | M1fs | | | | | | | | | matched |
| PBRAF- | | | A68fs*40 | | 67 | M | IV | 530 | Followup | Included | | un- |
| 201 | | | | | | | | | | | | matched |
| PBRAF- | | V157G | | | 66 | F | IV | 495 | Deceased | Included | | matched |
| 202 | | | | | | | | | | | | |
| PBRAF- | | L145P | | | 58 | M | IV | 315 | Deceased | Included | | matched |
| 203 | | | | | | | | | | | | |
| PBRAF- | | R196* | H66Sfs* | | 59 | F | IV | 992 | Followup | Included | | matched |
| 204 | | | 81 | | | | | | | | | |
| PBRAF- | | | | | 38 | M | IV | 502 | Deceased | Included | | matched |
| 205 | | | | | | | | | | | | |
| PBRAF- | | Y163N | H66fs*52 | | 70 | M | IIB | 841 | Deceased | Excluded | Initally | un- |
| 206 | | | | | | | | | | | pre- | matched |
| | | | | | | | | | | | sented | |
| | | | | | | | | | | | with | |
| PBRAF- | | | | | 74 | F | IV | 749 | Deceased | Included | | un- |
| 207 | | | | | | | | | | | | matched |
| PBRAF- | | C176Y | L94_ | | 67 | M | IIB | 239 | Deceased | Excluded | Initally | un- |
| 208 | | | G101del | | | | | | | | pre- | matched |
| | | | | | | | | | | | sented | |
| | | | | | | | | | | | with | |
| PBRAF- | | R273C | | | 64 | M | IIB | 365 | Deceased | Excluded | Initally | other |
| 209 | | | | | | | | | | | pre- | |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | | CDKN2A/ | SMAD4 | 71 | M | IV | 262 | Deceased | Included | | un- |
| 210 | | | B loss | splice site | | | | | | | | matched |
| PBRAF- | | c.375 + | | | 72 | F | IV | 722 | Deceased | Included | | un- |
| 211 | | 1G > C | | | | | | | | | | matched |
| PBRAF- | | R248Q | CDKN2A/ | | 67 | M | IV | 346 | Deceased | Included | | un- |
| 212 | | | B loss | | | | | | | | | matched |
| PBRAF- | | | | | 64 | M | I | 1050 | Followup | Excluded | Initally | other |
| 213 | | | | | | | | | | | pre- | |
| | | | | | | | | | | | sented | |
| | | | | | | | | | | | with | |
| | | | | | | | | | | | early | |
| | | | | | | | | | | | stage | |
| PBRAF- | | H193R | | | 46 | F | IV | 280 | Deceased | Included | | matched |
| 214 | | | | | | | | | | | | |
| PBRAF- | | X307_ | Y44Lfs* | | 66 | M | IV | 117 | Deceased | Included | | un- |
| 215 | | splice | 76 | | | | | | | | | matched |
| PBRAF- | | R273S | E88Cfs* | | 59 | F | III | 345 | Deceased | Excluded | Initally | un- |
| 216 | | | 34 | | | | | | | | pre- | matched |
| | | | | | | | | | | | sented | |
| | | | | | | | | | | | with | |
| PBRAF- | | R248W | L78fs*41 | | 55 | M | IIB | 655 | Followup | Excluded | Initally | un- |
| 217 | | | | | | | | | | | pre- | matched |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | | | | 59 | F | IV | 367 | Deceased | Included | | un- |
| 218 | | | | | | | | | | | | matched |
| PBRAF- | | | | | 60 | F | IV | 659 | Followup | Included | | un- |
| 301 | | | | | | | | | | | | matched |
| PBRAF- | | | CDKN2A/ | | 64 | M | IIB | 1079 | Deceased | Excluded | Initally | un- |
| 302 | | | B loss | | | | | | | | pre- | matched |
| | | | | | | | | | | | sented | |
| | | | | | | | | | | | with | |
| PBRAF- | | | CDKN2A/ | SMAD4 | 61 | F | IV | 1051 | Followup | Excluded | Re- | matched |
| 303 | | | B loss | R361_ | | | | | | | ceived | |
| | | | | C363del | | | | | | | an | |
| | | | | | | | | | | | NTRK | |
| | | | | | | | | | | | in- | |
| | | | | | | | | | | | hibitor | |
| | | | | | | | | | | | for an | |
| | | | | | | | | | | | NTRK | |
| PBRAF- | KRAS | | | | 71 | M | I | 240 | Followup | Excluded | Initally | other |
| 304 | G13D | | | | | | | | | | pre- | |
| | | | | | | | | | | | sented | |
| | | | | | | | | | | | with | |
| | | | | | | | | | | | early | |
| | | | | | | | | | | | stage | |
| PBRAF- | | | | | 55 | F | IIB | 1414 | Followup | Excluded | Initally | un- |
| 305 | | | | | | | | | | | pre- | matched |
| | | | | | | | | | | | sented | |
| | | | | | | | | | | | with | |
| PBRAF- | | | | SMAD4 | 55 | M | IV | 324 | Deceased | Included | | un- |
| 306 | | | | L536fs*1 | | | | | | | | matched |
| PBRAF- | | C141Y | | SMAD4 | 59 | F | IV | 579 | Deceased | Included | | matched |
| 307 | | | | S178* | | | | | | | | |
| PBRAF- | KRAS | | | | 68 | F | IV | 429 | Deceased | Included | | un- |
| 308 | G12V | | | | | | | | | | | matched |
| PBRAF- | KRAS | R175H | | SMAD2 | 85 | M | IV | 76 | Deceased | Included | | un- |
| 309 | G12D | | | R114H | | | | | | | | matched |
| PBRAF- | KRAS | E285K | | | 45 | M | IV | 559 | Followup | Included | | un- |
| 310 | am- | | | | | | | | | | | matched |
| | plified | | | | | | | | | | | |
| PBRAF- | KRAS | L97P | D415Efs* | | 75 | M | IIA | 523 | Followup | Excluded | Initally | other |
| 311 | C135Y | | 20; | | | | | | | | pre- | |
| | | | G423Lfs* | | | | | | | | sented | |
| | | | 1 | | | | | | | | with | |
| | | | | | | | | | | | non- | |
| | | | | | | | | | | | meta- | |
| | | | | | | | | | | | static | |
| PBRAF- | KRAS | V216M | | | 48 | M | IIB | 452 | Deceased | Excluded | Initally | un- |
| 312 | G12R | | | | | | | | | | pre- | matched |
| | | | | | | | | | | | sented | |
| | | | | | | | | | | | with | |
| PBRAF- | KRAS | | | | 68 | M | IV | 212 | Deceased | Excluded | No | other |
| 313 | G12D | | | | | | | | | | treat- | |
| | | | | | | | | | | | ment | |
| | | | | | | | | | | | docu- | |
| | | | | | | | | | | | mented | |
| | | | | | | | | | | | in the | |
| | | | | | | | | | | | ad- | |
| | | | | | | | | | | | vanced | |
| PBRAF- | KRAS | | | | 73 | F | I | 950 | Followup | Excluded | Initally | other |
| 314 | G12R | | | | | | | | | | pre- | |
| | | | | | | | | | | | sented | |
| | | | | | | | | | | | with | |
| | | | | | | | | | | | early | |
| | | | | | | | | | | | stage | |
| PBRAF- | KRAS | | | | 52 | F | IV | 2077 | Followup | Included | | un- |
| 315 | G12D | | | | | | | | | | | matched |
| PBRAF- | KRAS | R248Q | CDKN2A/ | | 86 | F | IV | 57 | Deceased | Included | | un- |
| 316 | G12R | | B loss | | | | | | | | | matched |
| PBRAF- | KRAS | T241P | A21Gfs* | SMAD4 | 68 | F | IV | 259 | Deceased | Included | | un- |
| 317 | G12V | | 14 | | | | | | | | | matched |
| PBRAF- | KRAS | F109_ | E33* | | 78 | F | IV | 374 | Followup | Included | | un- |
| 318 | G12V | L111- | | | | | | | | | | matched |
| | | delinsFL | | | | | | | | | | |
| PBRAF- | KRAS | G2455 | | | | F | IV | 899 | Deceased | Included | | matched |
| 319 | G12A | | | | | | | | | | | |
| PBRAF- | | E258K | CDKN2A/ | | 76 | F | IV | 732 | Deceased | Included | | un- |
| 320 | | | B loss | | | | | | | | | matched |
| PBRAF- | | | | | 62 | M | IV | 294 | Deceased | Included | | un- |
| 321 | | | | | | | | | | | | matched |
| PBRAF- | | | | | 56 | M | IV | 397 | Followup | Included | | un- |
| 401 | | | | | | | | | | | | matched |
| PBRAF- | | | | | 38 | F | IIB | 1043 | Deceased | Excluded | Initally | other |
| 402 | | | | | | | | | | | pre- | |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | | | | 70 | M | III | 1127 | Followup | Excluded | Initally | un- |
| 403 | | | | | | | | | | | pre- | matched |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | | | | 80 | M | IIB | 1020 | Followup | Excluded | Initally | |
| 404 | | | | | | | | | | | pre- | |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | E286K | F90L | SMAD4 | 86 | M | IIB | 750 | Followup | Excluded | Initally | |
| 405 | | | | R361H | | | | | | | pre- | |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | | | | 57 | F | IV | 568 | Followup | Included | | matched |
| 406 | | | | | | | | | | | | |
| PBRAF- | | V272L | | | 70 | F | IV | 684 | Deceased | Included | | matched |
| 407 | | | | | | | | | | | | |
| PBRAF- | | | | | 52 | F | IV | 1003 | Followup | Included | | matched |
| 408 | | | | | | | | | | | | |
| PBRAF- | | F134S | CDKN2A/ | SMAD4 | 75 | M | IV | 247 | Deceased | Included | | un- |
| 409 | | | B loss | S56fs*1 | | | | | | | | matched |
| PBRAF- | | | | | 61 | M | IIA | 2092 | Followup | Excluded | Initally | |
| 410 | | | | | | | | | | | pre- | |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | | CDKN2A/ | | 63 | M | IV | 112 | Followup | Included | | un- |
| 411 | | | B loss | | | | | | | | | matched |
| PBRAF- | | | | | 59 | M | IV | 282 | Deceased | Included | | un- |
| 412 | | | | | | | | | | | | matched |
| PBRAF- | | | | | 61 | M | IV | 1724 | Deceased | Included | | matched |
| 413 | | | | | | | | | | | | |
| PBRAF- | | | CDKN2A/ | SMAD4 | 57 | F | IV | 890 | Deceased | Included | | matched |
| 414 | | | B loss | splice site | | | | | | | | |
| | | | | 455-1G > C | | | | | | | | |
| PBRAF- | | | | | 56 | F | IV | 1055 | Deceased | Included | | un- |
| 415 | | | | | | | | | | | | matched |
| PBRAF- | | | | | 67 | F | IV | 176 | Deceased | Included | | un- |
| 416 | | | | | | | | | | | | matched |
| PBRAF- | | | CDKN2A/ | | 71 | M | IV | 655 | Deceased | Included | | un- |
| 417 | | | B loss | | | | | | | | | matched |
| PBRAF- | | | | SMAD4_A | 66 | M | IV | 735 | Deceased | Included | | un- |
| 418 | | | | 66fs*29 | | | | | | | | matched |
| PBRAF- | | | | | 45 | F | III | 863 | Deceased | Excluded | Initally | un- |
| 419 | | | | | | | | | | | pre- | matched |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | | | SMAD4 | 52 | M | IV | 496 | Deceased | Included | | matched |
| 420 | | | | Q534* | | | | | | | | |
| PBRAF- | | | | | 46 | M | IV | 552 | Deceased | Included | | un- |
| 421 | | | | | | | | | | | | matched |
| PBRAF- | | | CDKN2A/ | | 51 | F | III | 846 | Deceased | Excluded | Initally | un- |
| 422 | | | B loss | | | | | | | | pre- | matched |
| | | | | | | | | | | | sented | |
| | | | | | | | | | | | with | |
| | | | | | | | | | | | non- | |
| | | | | | | | | | | | meta- | |
| | | | | | | | | | | | static | |
| PBRAF- | | | | | 57 | F | IV | 999 | Followup | Included | | un- |
| 423 | | | | | | | | | | | | matched |
| PBRAF- | | | | | 72 | M | IV | 119 | Followup | Included | | un- |
| 424 | | | | | | | | | | | | matched |
| PBRAF- | | splice site | CDKN2A/ | | 42 | F | IV | 406 | Deceased | Included | | un- |
| 425 | | 1051_ | B loss | | | | | | | | | matched |
| | | 1100 + | | | | | | | | | | |
| | | 21del71 | | | | | | | | | | |

### Survival Analysis by BRAF Subgrouping

We performed exploratory survival analyses to assess the prognostic impact of each BRAF subgrouping (FIG. 2). These analyses across the “OS Cohort” included a subset of 54 patients with BRAF-mutated epithelial PC (see Table 2) who presented with metastatic disease (excludes 27 patients with IPMN's or resectable disease which have distinct prognostic implications compared to metastatic disease). Median overall survival was 1.9 years for the V600 subgroup (N=9), 1.9 years for the Fusion subgroup (N=18), 1.2 years for the Exon 11 subgroup (n=12), and Other 1.6 years (n=15). There were no significant differences in survival found between these categories of BRAF alterations (see FIG. 2).

MEK and RAE Inhibitors have Activity in Patients WIth KRAS Wild Type and BRAT-Mutated Pancreatic Cancers

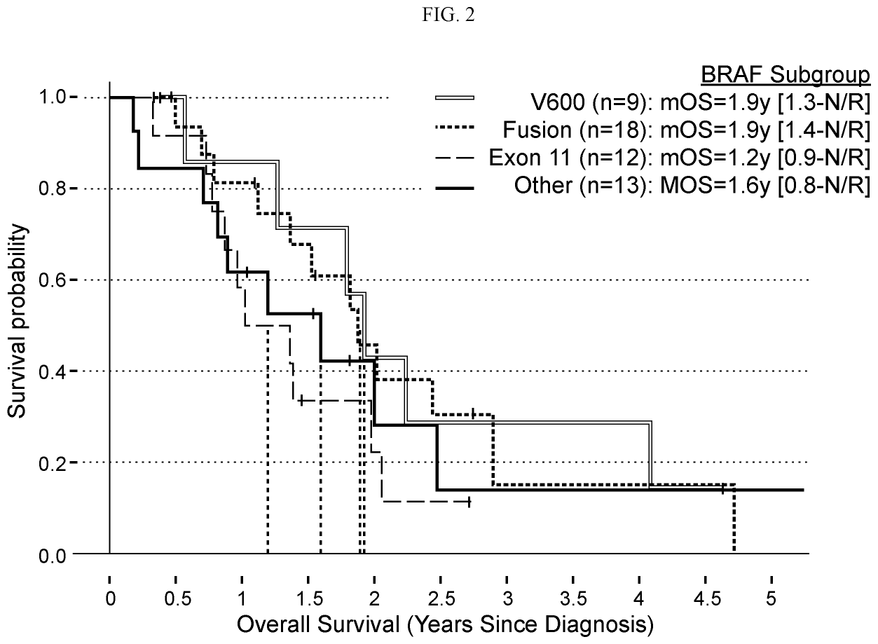

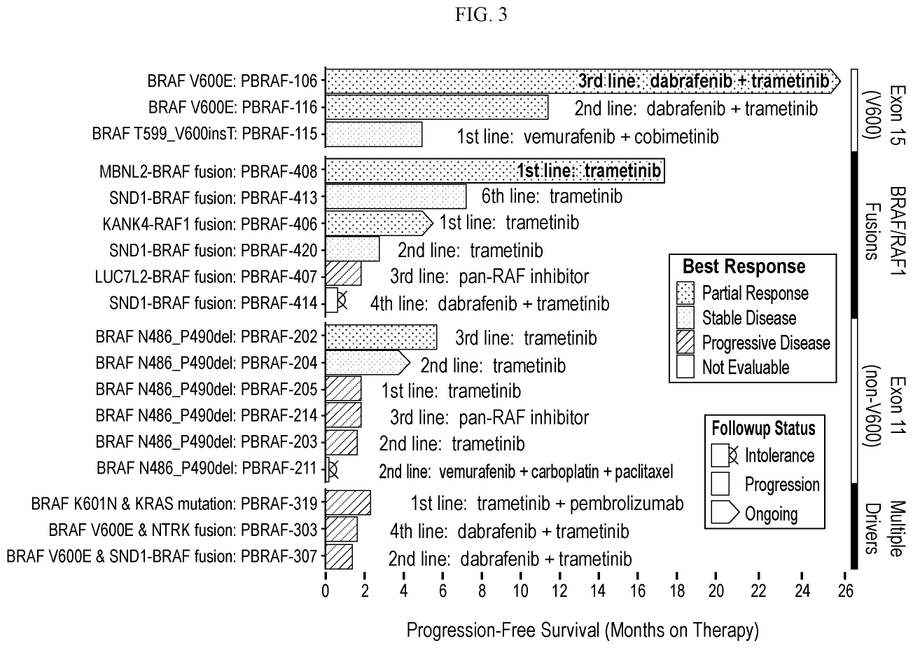

Response to BRAF targeted therapy was evaluated by the subgrouping described herein (FIG. 3). All 3 patients with exon 15 mutations had significant clinical benefit on dual BRAF/MEK targeted therapy. Of two with a V600E mutation one had a durable partial response for 48 weeks and another response ongoing after over 2 years. A patient with a non-canonical BRAF 1599 V600insT (also known as BRAF T599dup) achieved stable disease for approximately 20 weeks. In patients with fusion abnormalities, there was significant activity to single agent MEK inhibitors with 4/6 patients having disease control while on single agent trametinib including 2 with partial responses (one lasting 73 weeks and the other ongoing at 21 weeks) and 2 with stable disease (one lasting 30 weeks and the other 12 weeks). In patients who received a therapy matched to an Exon 11 mutation, 2/5 had therapeutic activity with one instance of stable disease continuing after 21 weeks and another with a partial response lasting 24 weeks (both on a single agent MEK inhibitor). One additional patient received vemurafenib plus chemotherapy (response not evaluable after initial toxicity and discontinuation) based on BRAF ΔNVTAP. Patients with confounding drivers or other BRAF alterations did not appear to derive any significant clinical benefit from targeted therapy consisting of MEK inhibitors given in combination with either a BRAF inhibitor (2/3) or immunotherapy (1/3) in this cohort (FIG. 3).

### Overall Survival Outcomes in Patients Receiving BRAF-Directed Therapy

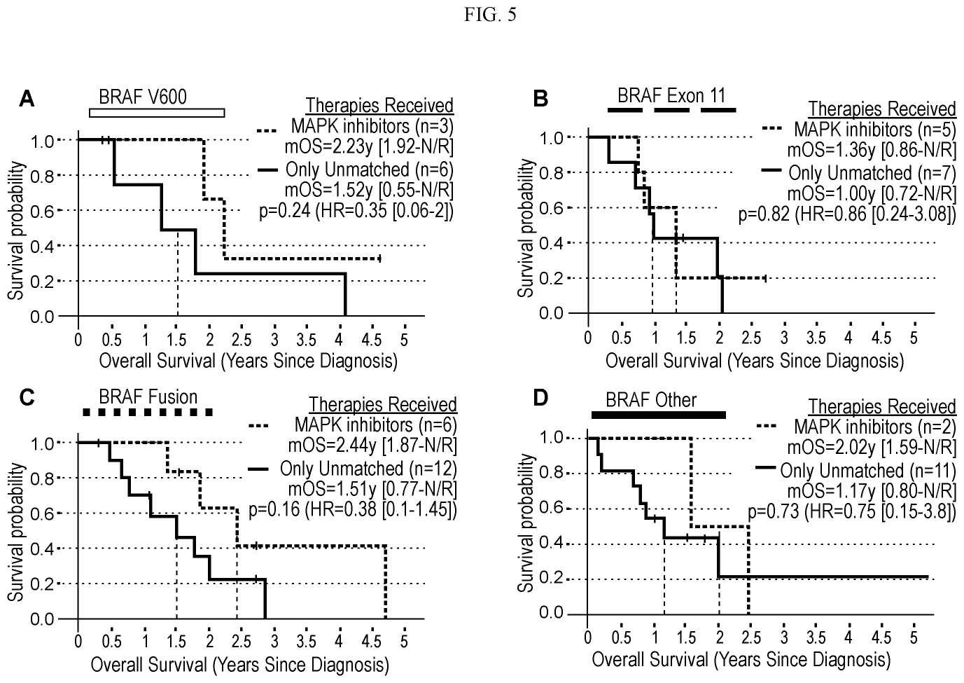

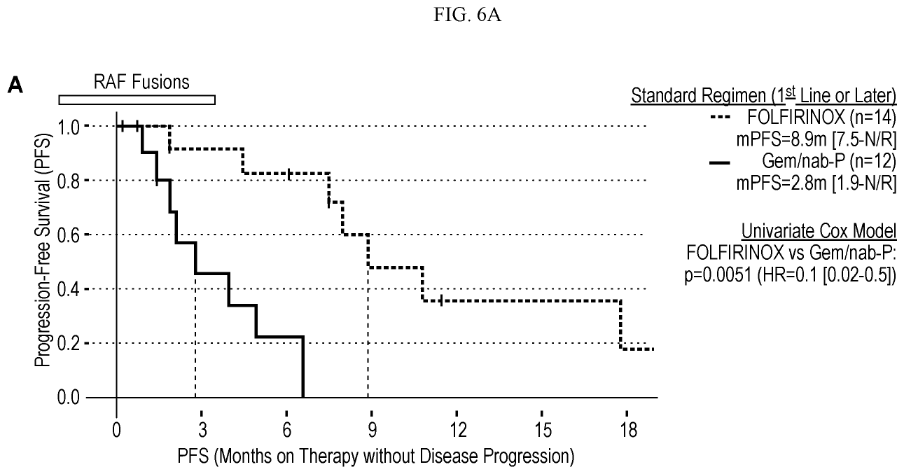

To better understand the possible real-world impact of BRAF-directed therapy on patient outcomes, we compared overall survival between patients with BRAF alterations (delineated by BRAF subclass) who received a molecularly-matched therapy targeting the MAPK signaling pathway (e.g. a BRAF, pan-RAF, MEK, or ERK inhibitor) vs patients with BRAF alterations who only received unmatched therapies (see Table 2 for baseline characteristics across the matched and unmatched cohorts). First, we assessed overall survival for each BRAF subclass individually (FIG. 5). Trends towards significance were stronger for exon 15 (p=0.24, HR=0.35 [0.06-2]) and fusions (p=0.16, HR=0.38 [0.1-1.45]) relative to exon 11 (p=0.82, HR=0.86 [0.24-3.08]) and other alterations (p=0.73, HR=0.75 [0.15-3.8]). Univariate Cox analysis across a combined cohort of patients with BRAF exon 15, exon 11, and fusion alterations was not significant but trending (p=0.07, HR=0.48 [0.21-1.07]) in favor of patients who received a matched therapy (mOS=1.9y [1.4-N/R], n=14) versus those who only received unmatched therapies (mOS=1.5y [0.9-2.9], n=25). Within the combined cohort of patients with BRAF exon 15 and fusions, we observed a similar trend which was modestly significant by log-rank test (p=0.04) but not by Cox regression (p=0.054 (HR=0.35 [0.12-1.02]) when comparing matched (mOS=2.4 [1.9-N/R], n=9) and unmatched (mOS=1.5y [1.1-N/R], n=18) groups.

### BRAF Alterations May Predict Response to Standard Chemotherapy

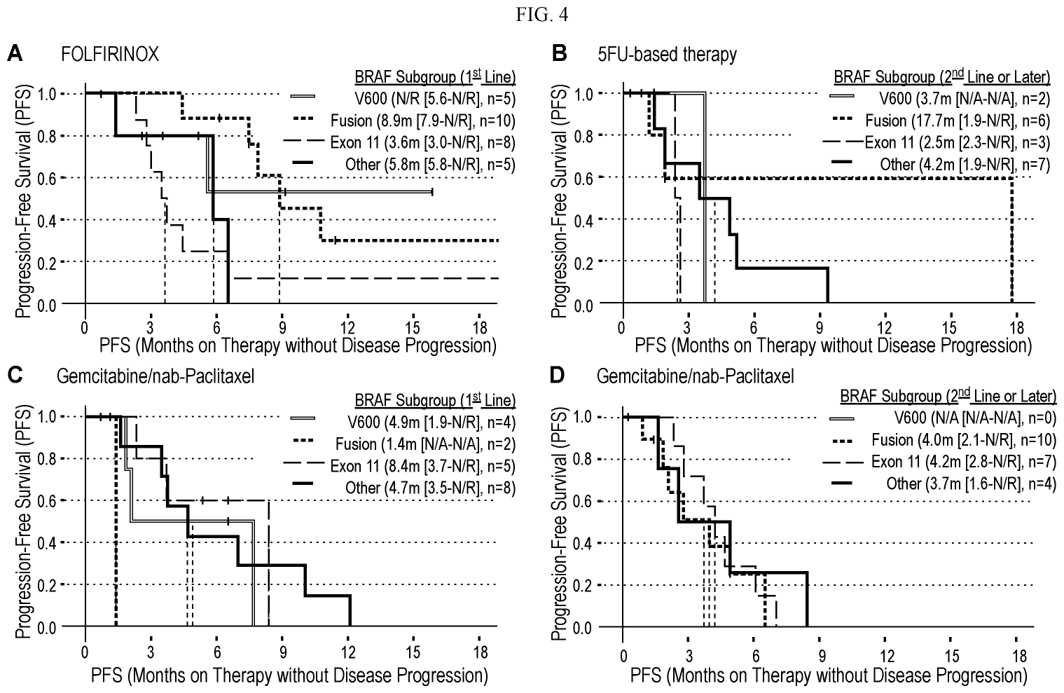

As an exploratory analysis, we evaluated median PFS for standard chemotherapy regimens across the clinical cohort and within each BRAF subgroup (FIG. 4). The two most common types of therapies documented in the advanced setting were gemcitabine plus nab-paclitaxel (Gem/nab-P, n=40) and 5-fluorouracil (5FU)-based regimens (n=46). In the 1^{st} line setting, patients with BRAF-mutated PC receiving FOLFIRINOX (FIG. 4A) or Gem/nab-P had a median PFS of 6.5 months [95% CI: 4.4-N/R] (n=28) or 4.7 months [95% CI: 2.3-N/R] (n=19), respectively. In subsequent lines of therapy (limit 1 per patient), 5FU-based therapies were implemented either as FOLFIRINOX (n=10), FOLFOX (n=2), FOLFIRI (n=2), or 5FU/nal-irinotecan (n=4) with an overall median PFS of 4.7 months [95% CI: 3.5-N/R] (FIG. 4B). Gemcitabine plus nab-paclitaxel given in 2^{nd} line had an overall median PFS of 4.0 months [95% CI: 2.8-6.5] (FIG. 4D).

We analyzed PFS outcomes in the 1^{st} line setting (FIG. 4A/4C) or in later lines (FIG. 4B/4D) for 5FU-based therapy (FIG. 4A/4B) or Gem/nab-P (FIG. 4C/4D) across each BRAF mutational subgroup. In patients receiving gemcitabine plus nab-paclitaxel, PFS did not significantly differ across subgroups. While these results are only considered exploratory, we observed a modest trend in favor of 5FU-based therapies that appeared to be specific to the Fusion subgroup.

In a follow-up analysis focusing on the Fusion cohort, we identified a significant difference in PFS (p=0.0051 (HR=0.1 [0.02-0.50]) between FOLFIRINOX (mPFS=8.9m [7.5-N/R], n=14, 1^{st} line or later) and Gem/nab-P (mPFS=2.8m [1.9-N/R], n=12, 1^{st} line or later) via univariate Cox regression (FIG. 6A). This subgroup analysis included only the patients who received the entire FOLFIRINOX regimen and these differences remained significant when applying a multivariate Cox model (p=0.027, HR=0.08 [0.01-0.75])) factoring in line of therapy (1′ line vs later lines: p=0.75, HR=1.34 [0.22-8.26]) (FIG. 6B) with or without a third term accounting for differences in adenocarcinoma versus acinar cell carcinoma histology (see FIG. 6 caption).

### Example 2

### Trial of Binimetinib in Combination with Encorafenib in Patients with Pancreatic Malignancies and a Somatic BRAF^{V600E} mutation

### Patient Eligibility

The following inclusion criteria are for purposes of the trial described herein. Performance of the embodiments of the present invention does not limit subject to these inclusion criteria. Pre-registration—Inclusion Criteria:
Age ≥18 years.
Histological confirmation of a pancreatic malignancy (including ampullary) as confirmed by the local pathology lab
Patients whose disease has progressed on (or who were intolerant of) at least one line of therapy for metastatic disease
Patients whose disease has recurred with metastatic disease ≤3 months of completion of neoadjuvant or adjuvant systemic chemotherapy; or patients with locally advanced disease whose disease progressed to metastatic disease on, or ≤3 months after completion of systemic chemotherapy would also be eligible
Provide informed written consent ≤28 days prior to registration.
Central electronic/paper confirmation of the presence of a BRAF^{V600E} mutation (Section 6.16). This review is mandatory prior to registration to confirm eligibility.
Results from a CLIA/CAP certified testing lab (commercial or institutional) that confirm the presence of a BRAF^{V600E} mutation in the patient's tumor must be submitted for central review.

The following registration criteria are for purposes of the trial described herein. Performance of the embodiments of the present invention does not limit subject to these registration criteria. Registration—Inclusion Criteria
Confirmation of the presence of BRAF′ mutation in the patient's tumor.
Measurable disease as defined herein.
ECOG Performance Status (PS) 0, 1, or 2.
The following laboratory values obtained ≥14 days prior to registration.
   Absolute neutrophil count (ANC)≥1500/mm³
   Platelet count ≥75,000/mm³
   Hemoglobin ≥9.0 g/dL
   Total bilirubin ≤1.5× upper limit of normal (ULN)
   Aspartate transaminase (AST) ≤2.5×ULN; in participants with liver metastases ≤5×ULN
   Aminotransferase (ALT) ≤2.5×ULN; in participants with liver metastases ≤5×ULN
   Calculated creatinine clearance must be ≥45 ml/min using the Cockcroft-Gault formula below:

$$\begin{matrix}\underline{Cockcroft - Gault Equation} : & \\ Creatinine clearance for males = & \frac{( 140 - age ) ( weight in kg )}{( 72 ) ( serum creatinine in mg / dL )} \\ Creatinine clearance for females = & \frac{( 140 - age ) ( weight in kg ) ( 0.85 )}{( 72 ) ( serum creatinine in mg / dL )}\end{matrix}$$

Negative pregnancy test done ≤7 days prior to registration, for women of childbearing potential only.
Willing to return to enrolling institution for follow-up (during the Active Monitoring Phase of the study).
Ability to swallow the investigational product tablets and capsules.
Willing to provide tissue and blood samples for correlative research purposes

The following registration criteria are for purposes of the trial described herein. Performance of the embodiments of the present invention does not necessarily exclude subjects having these registration criteria. Registration—Exclusion Criteria
Patients whose tumor harbors a BRAF non-V600E mutation or a BRAF fusion.
Prior therapy with BRAF inhibitor (e.g., encorafenib, dabrafenib, vemurafenib) and/or a
MEK inhibitor (e.g., binimetinib, trametinib, cobimetinib).
Known hypersensitivity or contraindication to any component of binimetinib or encorafenib or their excipients.
Any of the following because this study involves an investigational agent whose genotoxic, mutagenic and teratogenic effects on the developing fetus and newborn are unknown: Pregnant women, Nursing women, Men or women of childbearing potential who are unwilling to employ adequate contraception.
Co-morbid systemic illnesses or other severe concurrent disease which, in the judgment of the investigator, would make the patient inappropriate for entry into this study or interfere significantly with the proper assessment of safety and toxicity of the prescribed regimens.
Immunocompromised patients and patients known to be HIV positive and currently receiving antiretroviral therapy. NOTE: Patients known to be HIV positive, but without clinical evidence of an immunocompromised state, are eligible for this trial.
Uncontrolled intercurrent illness including, but not limited to, ongoing or active infection, symptomatic congestive heart failure, cardiac arrhythmia, or psychiatric illness/social situations that would limit compliance with study requirements.
History of acute coronary syndromes (including myocardial infarction, unstable angina, coronary artery bypass grafting, coronary angioplasty or stenting)<6 months prior to registration.
Left ventricular ejection fraction (LVEF)≤50% as determined by MUGA or ECHO.
Uncontrolled hypertension defined as persistent systolic blood pressure ≥150/100 mmHg or diastolic blood pressure ≥100 mmHg despite current therapy.
Triplicate average baseline QTc interval ≥480 ms.
Receiving any other investigational agent which would be considered as a treatment for the primary neoplasm.
Patients who have had another active malignancy within the past two years are ineligible
EXCEPT FOR patients with cervical cancer in situ, in situ carcinoma of the bladder, non-melanoma carcinoma of the skin, or patients who have had therapy with curative intent for breast or prostate cancer, but remain on adjuvant hormonal therapy.
Received anticancer therapy including chemotherapy, immunotherapy, or antineoplastic biologic therapy (e.g., erlotinib, cetuximab, bevacizumab etc.), ≤14 days (≤28 days for an antibody-based therapy) prior to registration.
Patients who have undergone major surgery (e.g., in-patient procedures)≤6 weeks prior to registration or who have not recovered from side effects of such procedure.
Patients who have had radiotherapy ≤14 days prior to registration or who have not recovered from side effects of such procedure. NOTE: Palliative radiation therapy must be complete 7 days prior to the first dose of study treatment.
Patient has not recovered to ≤Grade 2 from toxic effects of prior therapy before registration. EXCEPTIONS: Stable chronic conditions (≤Grade 1) that are not expected to resolve (such as neuropathy, myalgia, alopecia, prior therapy-related endocrinopathies).
Uncontrolled or symptomatic brain metastases or leptomeningeal carcinomatosis that are not stable, require steroids, are potentially life-threatening or have required radiation ≤28 days prior to registration.
Impairment of gastrointestinal function or disease which may significantly alter the absorption of study drug (e.g., active ulcerative disease, uncontrolled vomiting or diarrhea, malabsorption syndrome, small bowel resection with decreased intestinal absorption), or recent (≤12 weeks) history of a partial or complete bowel obstruction, or other condition that will interfere significantly with the absorption or oral drugs.
Known history of acute or chronic pancreatitis.
Concurrent neuromuscular disorder that is associated with elevated CK (e.g., inflammatory myopathies, muscular dystrophy, amytrophic lateral sclerosis, spinal muscular atrophy).
History or current evidence of RVO or current risk factors for RVO (e.g., uncontrolled glaucoma or ocular hypertension, history of hyperviscosity or hypercoagulability syndromes); history of retinal degenerative disease.
Current use of prohibited medication (including herbal medications, supplements, or foods), as described herein, or use of prohibited medication ≤7 days prior to registration.
History of thromboembolic or cerebrovascular events ≤12 weeks prior to registration. Examples include transient ischemic attacks, cerebrovascular accidents, hemodynamically significant (i.e. massive or sub-massive) deep vein thrombosis or pulmonary emboli.
Evidence of Hepatitis B Virus (HBV) or Hepatitis C Virus (HCV) infection.

### Test Schedule

| | | | | | Every | | | |
|---|---|---|---|---|---|---|---|---|
| | | | Baseline | Every 14 | 28 days | | | 30 |
| | | | (Prior to | days for | (+/− 3 | Prior | | Days |
| | | ≤14 days | Cycle 1 | the first | days) | to 3^{rd} and | At PD, | after |
| Tests and | | prior | Day 1 | cycle (28 | after | subsequent | withdrawal | last |
| procedures | Pre-reg | to reg | treatment) | days) | Cycle 1 | odd cycles | or removal | dose |
| History and exam, | | X | | X | X | | X | |
| wt, ECOG PS⁶ | | | | | | | | |
| Height | | X | | | | | | |
| Adverse event | | | | X | X | | X | X⁹ |
| assessment | | | | | | | | |
| Informed Consent | X | | | | | | | |
| ECHO/MUGA^{R, 10} | | X | | | X | | X | |
| Hematology: CBC/ | | X | X | X | X | | X | |
| differential | | | | | | | | |
| Chemistry: AST, | | X | X | X | X | | X | |
| ALT, alk phos, T. | | | | | | | | |
| bili, creatinine, | | | | | | | | |
| calcium, glucose, | | | | | | | | |
| Na, K | | | | | | | | |
| CPK and troponin¹¹ | | | X | | X | | | |
| Ophthalmic exam ^{R, 5} | | X | | | | | X | |
| Imaging (CT/PET | | X¹ | | | | X¹ | X¹ | |
| CT or MRI) and | | | | | | | | |
| tumor measurement | | | | | | | | |
| Urine pregnancy | | X | | | | | | |
| test² | | | | | | | | |
| Central | X | | | | | | | |
| electronic/paper | | | | | | | | |
| confirmation of a | | | | | | | | |
| BRAF^{V600E} | | | | | | | | |
| mutation⁷ | | | | | | | | |
| Mandatory tissue | | | X | | | | | |
| sample (see Section | | | | | | | | |
| 17.0)^{R, 8} | | | | | | | | |
| Mandatory blood | | | X | | X | X | X | |
| sample (see Section | | | | | | | | |
| 14.0)^{R, 4} | | | | | | | | |
| Patient Medication | | | | | X | | X | |
| Diary (Appendix I)³ | | | | | | | | |

¹CT scans-chest, abdomen, and pelvis with IV and PO contrast; in the case of iodine contrast allergies an MRI of the abdomen and pelvis with gadolinium + a non-contrast chest CT is appropriate. Use same imaging throughout the study. Imaging at PD should only be repeated if not done within 28 days prior.
²For women of childbearing potential only. Must be done ≤ 7 days prior to registration.
³The diary must begin the day the patient starts taking the medication and must be completed per protocol and returned to the treating institution OR compliance must be documented in the medical record by any member of the care team.
⁴Streck tubes are required for this collection.
⁵ A full ophthalmic exam will be performed by an ophthalmologist at screening, as needed during on-study treatment phase and at end of treatment, and include best corrected visual acuity, slit lamp examination, intraocular pressure, dilated fundoscopy and Ocular Coherence Tomography (OCT). Examination of the retina is required, especially to identify findings associated with serous retinopathy and RVO.
⁶ After baseline, patients receiving binimetinib should be assessed at every physical examination for decreased visual acuity using a gross perimetry test (as opposed to automated visual field testing). Symptomatic patients should be referred for a full ophthalmic consultation.
⁷Results from a CLIA/CAP certified testing lab (commercial or institutional) that confirm the presence of a BRAF^{V600E} mutation in the patient's tumor must be submitted for central review. See section 6.16 for detailed instructions.
8Receipt of archival tumor tissue is not required for study registration and initiation of therapy. However, it is mandatory to receive the required tissue within 30 days from registration. See section 17.0.
⁹The 30 days after last dose of treatment evaluation can be done over the phone.
¹⁰ Scans are to be performed at screening, on Cycle 2 Day 1 and Cycle 5 Day 1, then every 12 weeks and end of treatment.
¹¹CPK and Troponin levels to be completed Cycle 1 Day 1 and Cycle 2 Day 1.

### Protocol Treatment

Treatment Schedule—Starting Day 1 of Cycle 1 (28 day Cycles), patient will administer: Encorafenib 450 mg orally daily, Binimetinib 45 mg orally twice daily.

### Dosage Modification Based on Adverse Events

Strictly follow the modifications in this table for the first two cycles, until individual treatment tolerance can be ascertained. Thereafter, these modifications should be regarded as guidelines to produce mild-to-moderate, but not debilitating, side effects. If multiple adverse events are seen, administer dose based on greatest reduction required for any single adverse event observed. Reductions or increases apply to treatment given in the preceding cycle and are based on adverse events observed since the prior dose.

### Dose Levels (Based on Adverse Events), if Applicable

| Dose Level | Encorafenib | Binimetinib |
|---|---|---|
| 0 | 450 mg | 45 mg |
| −1 | 300 mg | 30 mg |
| −2 | 225 mg | 15 mg |

* Dose level 0 refers to the starting dose.

The lowest recommended dose level of encorafenib is 225 mg QD and the lowest recommended dose level of binimetinib is 15 mg BID. When the AE that resulted in a dose reduction improves to and remains stable at the patient's baseline level for a minimum of 14 days, the dose can be re-escalated to the next dose level at the discretion of the Investigator, provided there are no other concomitant toxicities that would prevent drug re-escalation. There is no limit to the number of times the patient can have their dose reduced or re-escalated, however:
No dose re-escalation of encorafenib is allowed after a dose reduction due to prolonged
QTcF ≥501 msec
No dose re-escalation of binimetinib is allowed after a dose reduction due to LVEF dysfunction
No dose re-escalation of binimetinib or encorafenib is allowed after a dose reduction due to retinal toxicity ≥Grade 2.
If binimetinib is withheld, consider reducing encorafenib to a maximum dose of 300 mg daily until binimetinib is resumed.

### Recommended Encorafenib Dose Modifications

| | | |
|---|---|---|
| CTCAE | | |
| System/Organ/Class (SOC) | ADVERSE EVENT | ACTION** |

BASED ON INTERVAL ADVERSE EVENT

| | | |
|---|---|---|
| | Non-cutaneous RAS | Permanently discontinue |
| | mutation-positive | |
| | malignancies | |
| Eye Disorder | Uveitis | |
| | Grade 1-3 | If Grade 1 or 2 does not respond to specific ocular |
| | | therapy, or for Grade 3 uveitis, withhold |
| | | encorafenib and binimetinib for up to 6 weeks |
| | Grade 4 | Permanently discontinue encorafenib and |
| | | binimetinib |
| | Eye disorders-Other, | |
| | specify | |
| | Grade 1-2 | Maintain dose level of encorafenib and binimetinib |
| | | and increase frequency of ophthalmic monitoring |
| | | to at least every 14 days until stabilization or |
| | | resolution. |
| | Grade 3 | Interrupt dosing of encorafenib and binimetinib and |
| | | refer patient to ophthalmologist within 7 days. |
| | | If resolved to Grade ≤1 in ≤21 days, |
| | | resume treatment at 1 reduced dose level of |
| | | encorafenib and binimetinib. |
| | | If not resolved to Grade ≤1 in ≤21 days, |
| | | permanently discontinue encorafenib and |
| | | binimetinib and close follow-up with |
| | | ophthalmic monitoring until stabilization or |
| | | resolution. |
| | Grade 4 | Permanently discontinue encorafenib and |
| | | binimetinib and immediate follow-up with |
| | | ophthalmic monitoring until stabilization or |
| | | resolution. |
| Investigations | Electrocardiogram | |
| | QT corrected interval | |
| | prolonged | |
| | QTcF >500 ms and ≤60 | 1^{st} occurrence: |
| | ms increase from | Temporarily interrupt dosing of |
| | baseline | encorafenib until QTcF <500 ms. Then |
| | | resume treatment at 1 reduced dose level of |
| | | encorafenib. |
| | | 2^{nd} occurrence: |
| | | Temporarily interrupt dosing of |
| | | encorafenib treatment until QTcF <500 |
| | | ms. Then resume treatment at 1 reduced |
| | | dose level of encorafenib. |
| | | 3^{rd} occurrence: |
| | | Permanently discontinue encorafenib. |
| | QTcF >500 ms and >60 | Permanently discontinue encorafenib. |
| | ms increase from | |
| | baseline | |
| | Alanine | |
| | aminotransferase or | |
| | aspartate | |
| | aminotransferase | |
| | increased | |
| | Grade 2 | Maintain encorafenib dose. |
| | | If no improvement within 4 weeks, |
| | | withhold encorafenib until improved to |
| | | Grade 0-1 or to pretreatment/baseline levels |
| | | and then resume at the same dose. |
| | Grade 3 or 4 | See Other Adverse Reactions |
| | Investigations-Other, | |
| | specify | |
| | Recurrent Grade 2 or | Withhold for up to 4 weeks. |
| | First occurrence of | If improves to Grade 0-1 or to |
| | any Grade 3 | pretreatment/baseline levels, resume at |
| | | reduced dose. |
| | | If no improvement, permanently |
| | | discontinue encorafenib. |
| | First occurrence of ay | Withhold for up to 4 weeks. |
| | Grade 4 | If improves to Grade 0-1 or to |
| | | pretreatment/baseline levels, then resume at |
| | | a reduced dose. |
| | | If no improvement, permanently |
| | | discontinue encorafenib. |
| | Recurrent Grade 3 | Consider permanently discontinuing encorafenib |
| | | and binimetinib. |
| | Recurrent Grade 4 | Permanently discontinue encorafenib and |
| | | binimetinib. |
| Skin and | Palmar-plantar | |
| subcutaneous tissue | erythrodysesthesia | |
| disorders | syndrome | |
| | Grade 1 | Maintain dose of encorafenib. Promptly institute |
| | | supportive measures, such as topical therapy, for |
| | | symptomatic relief. Give instruction on life-style |
| | | modifications. |
| | Grade 2 | 1^{st} occurrence: |
| | | Maintain dose of encorafenib and HFSR |
| | | should be closely monitored. Promptly |
| | | institute supportive measures, such as |
| | | topical therapy, for symptomatic relief. |
| | | Give instruction on life-style modifications. |
| | | If no improvement ≤14 days, interrupt |
| | | dosing of encorafenib until resolved to |
| | | Grade ≤1. Resume treatment with |
| | | encorafenib at current dose level. Continue |
| | | supportive measures, such as topical |
| | | therapy, for symptomatic relief. Give |
| | | instruction on life-style modifications. |
| | | Additional occurrence: |
| | | Treatment with encorafenib may be |
| | | maintained or interrupted based upon the |
| | | Investigator's discretion. Continue |
| | | supportive measures, such as topical |
| | | therapy, for symptomatic relief. Give |
| | | instruction on life-style modifications. |
| | | If interrupted dosing of encorafenib per |
| | | Investigator's judgment, interrupt until resolved to |
| | | Grade ≤1. Resume treatment with encorafenib at |
| | | the same dose level or 1 reduced dose level based |
| | | upon the Investigator's discretion. |
| | Grade 3 | 1^{st} or additional occurrence: |
| | | Interrupt dosing of encorafenib until |
| | | resolved to Grade ≤1. Promptly initiate |
| | | supportive measures, such as topical |
| | | therapy, for symptomatic relief. Give |
| | | instruction on life-style modifications. |
| | | Reassess the patient weekly. Then resume |
| | | treatment at one reduced dose level of |
| | | encorafenib. |
| | | Consider referral to dermatologist and |
| | | manage HFSR per dermatologist's |
| | | recommendation. |
| | | >3^{nd} occurrence: |
| | | Interrupt dosing of encorafenib until resolved to |
| | | Grade ≤1, decision to resume treatment with |
| | | encorafenib at one reduced dose level or |
| | | permanently discontinue encorafenib should be |
| | | based upon the Investigator's discretion. |
| | Skin and | |
| | subcutaneous tissue | |
| | disorders-Other, | |
| | specify | |
| | Grade 2 | If no improvement within 2 weeks, withhold until |
| | | Grade 0-1. Resume at same dose if first occurrence |
| | | or reduce dose if recurrent. |
| | Grade 3 | Withhold until Grade 0-1. Resume at same dose if |
| | | first occurrence or reduce dose if recurrent. |
| | Grade 4 | Permanently discontinue encorafenib. |
| Gastrointestinal | Nausea/Vomiting | |
| disorders | Grade 1-2 | Maintain dose level of encorafenib and |
| | | binimetinib. Promptly institute antiemetic measure. |
| | Grade 3 | Interrupt dosing of encorafenib and binimetinib |
| | | until resolved to Grade ≤1. Then resume treatment |
| | | at 1 reduced dose level of encorafenib. Resume |
| | | treatment with binimetinib at the current dose if, in |
| | | the judgment of the Investigator, the toxicity is |
| | | considered to be unrelated to binimetinib, or at 1 |
| | | reduced dose level. |
| | | Note: Interrupt dosing of encorafenib and |
| | | binimetinib for ≥Grade 3 vomiting or Grade 3 |
| | | nausea only if the vomiting or nausea cannot be |
| | | controlled with optimal antiemetics (as per local |
| | | practice). |
| | Grade 4 | Permanently discontinue encorafenib and |
| | | binimetinib. |

* Located at ctep.cancer.gov/protocolDevelopment/electronic_applications/ctc.htm
**Use the following to describe actions in the Action column:
Modified = A modification indicates a change in dose level during the current cycle. However, if a modification was issued after the last dose was received in the current cycle, the modification should be reported on the subsequent cycle. “Yes, planned” should be selected if the dose level was changed according to protocol guidelines (i.e. due to adverse events, lab values, etc.); “Yes, unplanned” should be selected if the dose level change was not a part of protocol guidelines (e.g. vacation, mistake, etc.). Held doses (including omissions and delays) should not be reported as modifications.
Omitted = An omission indicates a dose was skipped and not made up, or that the drug was discontinued.
Delayed = A delay indicates dose was postponed or not received when expected.

Recommended Binimetinib Dose Modifications

| | | |
|---|---|---|
| CTCAE | | |
| System/Organ/Class (SOC) | ADVERSE EVENT | ACTION** |

BASED ON INTERVAL ADVERSE EVENT

| | | |
|---|---|---|
| Eye Disorders | Retinopathy and | |
| | Retinal detachment | |
| | Grade 2 or above | Withhold binimetinib for up to 10 days. |
| | | If improves and becomes asymptomatic, |
| | | resume at the same dose. |
| | | If not improved, resume at a lower dose |
| | | level or permanently discontinue |
| | | binimetinib. |
| | Retinal Vein | |
| | Occlusion (RVO) | |
| | Eye disorders-Other, | |
| | specify | |
| | Grade 1-2 | Maintain dose level of encorafenib and binimetinib |
| | | and increase frequency of ophthalmic monitoring |
| | | to at least every 14 days until stabilization or |
| | | resolution. |
| | Grade 3 | Interrupt dosing of encorafenib and binimetinib and |
| | | refer patient to ophthalmologist within 7 days. |
| | | If resolved to Grade ≤1 in ≤21 days, |
| | | resume treatment at 1 reduced dose level of |
| | | encorafenib and binimetinib. |
| | | If not resolved to Grade ≤1 in ≤21 days, |
| | | permanently discontinue encorafenib and |
| | | binimetinib and close follow-up with |
| | | ophthalmic monitoring until stabilization or |
| | | resolution. |
| | Grade 4 | Permanently discontinue encorafenib and |
| | | binimetinib and immediate follow-up with |
| | | ophthalmic monitoring until stabilization or |
| | | resolution. |
| | Uveitis | |
| | Grade 1-3 | If Grade 1 or 2 does not respond to specific ocular |
| | | therapy, or for Grade 3 uveitis, withhold |
| | | binimetinib and encorafenib for up to 6 weeks. |
| | | If improved, resume at same or reduced |
| | | dose. |
| | | If not improved, permanently discontinue |
| | | binimetinib. |
| | Grade 4 | Permanently discontinue encorafenib and |
| | | binimetinib. |
| Cardiac Disorders | Heart Failure and Left | |
| | ventricular systolic | |
| | dysfunction | |
| | Grade 1 | Withhold binimetinib for up to 4 weeks, evaluate |
| | | LVEF every 2 weeks. |
| | | Resume binimetinib at a reduced dose if the |
| | | following are present: |
| | | LVEF is at or above the LLN and |
| | | Absolute decrease from baseline is 10% or |
| | | less and |
| | | Patient is asymptomatic. |
| | | If LVEF does not recover within 4 weeks |
| | | permanently discontinue binimetinib. |
| | Grade 3-4 | Permanently discontinue binimetinib. Closely |
| | | monitor LVEF until resolution or up to 16 weeks. |
| Respiratory, | Pneumonitis, | |
| thoracic and | pulmonary fibrosis | |
| mediastinal | Grade 2 | Withhold binimetinib for up to 4 weeks. |
| disorders | | If improves to Grade 0-1, resume at a |
| | | reduced dose. |
| | | If not resolved within 4 weeks, |
| | | permanently discontinue. |
| | Grade 3 or Grade 4 | Permanently discontinue binimetinib. |
| Investigations | Alanine | |
| | aminotransferase or | |
| | aspartate | |
| | aminotransferase | |
| | increased | |
| | Grade 2 | Maintain binimetinib dose. |
| | | If no improvement within 2 weeks, |
| | | withhold binimetinib until improved to |
| | | Grade 0-1 or to pretreatment/baseline levels |
| | | and then resume at the same dose. |
| | Grade 3 or 4 | See Other Adverse Reactions |
| | CPK increased | |
| | Grade 4 | Withhold binimetinib dose for up to 4 weeks. |
| | asymptomatic or Any | If improved to Grade 0-1 resume at a |
| | Grade with symptoms | reduced dose. |
| | or with renal | If not resolved within 4 weeks, |
| | impairment | permanently discontinue binimetinib. |
| | Investigations-Other, | |
| | specify | |
| | Recurrent Grade 2 or | Withhold for up to 4 weeks. |
| | First occurrence of | If improves to Grade 0-1 or to |
| | any Grade 3 | pretreatment/baseline levels, resume at |
| | | reduced dose. |
| | | If no improvement, permanently |
| | | discontinue encorafenib and binimetinib. |
| | First occurrence of | Permanently discontinue or withhold for up to 4 |
| | any Grade 4 | weeks. |
| | | If improves to Grade 0-1 or to |
| | | pretreatment/baseline levels, then resume at |
| | | a reduced dose. |
| | | If no improvement, permanently |
| | | discontinue encorafenib and binimetinib. |
| | Recurrent Grade 3 | Consider permanently discontinuing encorafenib |
| | | and binimetinib. |
| | Recurrent Grade 4 | Permanently discontinue. |
| Gastrointestinal | Nausea/Vomiting | |
| disorders | Grade 1-2 | Maintain dose level of encorafenib and |
| | | binimetinib. Promptly institute antiemetic measure. |
| | Grade 3 | Interrupt dosing of encorafenib and binimetinib |
| | | until resolved to Grade ≤1. Then resume treatment |
| | | as 1 reduced dose level of encorafenib. Resume |
| | | treatment with binimetinib at the current dose if, in |
| | | the judgement of the Investigator, the toxicity is |
| | | considered to be unrelated to binimetinib, or at 1 |
| | | reduced dose level. |
| | | Note: Interrupt dosing of encorafenib and |
| | | binimetinib for ≥Grade 3 vomiting or Grade 3 |
| | | nausea only if the vomiting or nausea cannot be |
| | | controlled with optimal antiemetics (as per local |
| | | practice). |
| | Grade 4 | Permanently discontinue encorafenib and |
| | | binimetinib. |
| Skin and | Skin and | |
| subcutaneous tissue | subcutaneous tissue | |
| disorders | disorders-Other, | |
| | specify | |
| | Grade 2 | If no improvement within 2 weeks, withhold until |
| | | Grade 0-1. Resume at same dose if first occurrence |
| | | or reduce dose if recurrent. |
| | Grade 3 | Withhold until Grade 0-1. Resume at same dose if |
| | | first occurrence or reduce dose if recurrent. |
| | Grade 4 | Permanently discontinue encorafenib and |
| | | binimetinib. |

* Located at ctep.cancer.gov/protocolDevelopment/electronic_applications/ctc.htm
**Use the following to describe actions in the Action column:
Modified = A modification indicates a change in dose level during the current cycle. However, if a modification was issued after the last dose was received in the current cycle, the modification should be reported on the subsequent cycle. “Yes, planned” should be selected if the dose level was changed according to protocol guidelines (i.e. due to adverse events, lab values, etc.); “Yes, unplanned” should be selected if the dose level change was not a part of protocol guidelines (e.g. vacation, mistake, etc.). Held doses (including omissions and delays) should not be reported as modifications.
Omitted = An omission indicates a dose was skipped and not made up, or that the drug was discontinued.
Delayed = A delay indicates dose was postponed or not received when expected.

### Ancillary Treatment/Supportive Care

Antiemetics may be used at the discretion of the attending physician.
Blood products and growth factors should be utilized as clinically warranted and following institutional policies and recommendations. The use of growth factors should follow published guidelines of the American Society of Clinical Oncology (ASCO), Recommendations for the Use of WBC Growth Factors: American Society of Clinical Oncology Clinical Practice Guideline Update. J Clin Oncol 2015; 33:3199-3212.
Patients should receive full supportive care while on this study. This includes blood product support, antibiotic treatment, and treatment of other newly diagnosed or concurrent medical conditions. All blood products and concomitant medications such as antidiarrheals, analgesics, and/or antiemetics received from the first day of study treatment administration until 30 days after the final dose will be recorded in the medical records.
Diarrhea: This could be managed conservatively with loperamide. The recommended dose of loperamide is 4 mg at first onset, followed by 2 mg every 2-4 hours until diarrhea free (maximum 16 mg/day).
In the event of grade 3 or 4 diarrhea, the following supportive measures are allowed: hydration, octreotide, and antidiarrheals. If diarrhea is severe (requiring intravenous rehydration) and/or associated with fever or severe neutropenia (grade 3 or 4), broad-spectrum antibiotics must be prescribed. Patients with severe diarrhea or any diarrhea associated with severe nausea or vomiting should be hospitalized for intravenous hydration and correction of electrolyte imbalances.
Permitted Concomitant Therapy Requiring Caution and/or Action Encorafenib is a reversible inhibitor of CYP1A2, CYP2B6, CYP2C8, CYP2C9, CYP3A4 and UGT1A1. It is also a time-dependent inhibitor of CYP3A4, and induced CYP2B6, CYP2C9 and CYP3A4 in human primary hepatocytes. Permitted medications to be used with caution in this study include those that are sensitive substrates of CYP1A2, CYP2B6, CYP2C9, CYP2C9, CYP3A4, and UGT1A1 or those substrates that have a narrow therapeutic index.
There is a potential for encorafenib to induce CYP3A4, which may reduce the effectiveness of hormonal contraception methods. Therefore, the use of at least 1 form of non-hormonal contraception is required for females of childbearing potential during participation in this study. Caution should be used in participants receiving concomitant treatment with other drugs that are substrates of CYP3A4 as the efficacy of these drugs could be reduced when administered with encorafenib.
Encorafenib has been identified in vitro to be metabolized by CYP3A4 and to a lesser extent by CYP2C19. The use of strong inhibitors of CYP3A4 is prohibited. Concomitant use of moderate CYP3A4 inhibitors while on study should be avoided. If use of moderate CYP3A4 inhibitors is unavoidable and no alternatives are available, short-term use (≤30 days) is permitted with accompanying dose reduction to one-half of the encorafenib dose prior to use of moderate CYP3A4 inhibitors (or as close as can be achieved without exceeding the target dose). The encorafenib dose that was taken prior to initiating the CYP3A4 inhibitor may be resumed after the inhibitor has been discontinued for 3 to 5 elimination half-lives. Strong inhibitors of CYP2C19 should be used with caution when co-administered with encorafenib. Use of moderate and strong inducers of CYP3A4 is prohibited.
In vitro data showed that encorafenib is a substrate of the transporter P-gp. Thus, drugs that are known to inhibit or induce P-gp should be used with caution. Encorafenib is also a potent inhibitor of the renal transporters, OAT1, OAT3 and OCT2, and the hepatic transporters OATP1B1 and OATP1B3. The co-administration of drugs that are known to be sensitive or narrow therapeutic index substrates of OAT1, OAT3, OCT2, OATP1B1 or OATP1B3 should be used with caution.
In vitro, binimetinib has been identified to be primarily metabolized by glucuronidation through UGT1A1. Binimetinib has also been shown to be a substrate of P-gp and BCRP. It is advised that inhibitors and inducers of UGT1A1, P-gp or BCRP transporters should be taken with caution when co-administered with binimetinib.
Prohibited Concomitant Therapy—Concomitant strong systemic CYP3A4 inhibitors and strong or moderate systemic CYP3A4 inducers are likely to significantly increase or decrease encorafenib exposure, respectively, and thus should not be used during this study.

### Treatment Evaluation Using RECIST Guideline

Response and progression will be evaluated in this study using the new international criteria proposed by the revised Response Evaluation Criteria in Solid Tumors (RECIST) guidelines (version 1.1). Changes in the largest diameter (unidimensional measurement) of the tumor lesions and the short axis measurements in the case of lymph nodes are used in the RECIST guideline (Eisenhauer et al, 2009).

Schedule of Evaluations: For the purposes of this study, patients should be reevaluated every 8 weeks. In addition to a baseline scan, confirmatory scans should also be obtained not less than 4 weeks following initial documentation of objective response.

### Definitions of Measurable and Non Measurable Disease

Measurable Disease—
A non-nodal lesion is considered measurable if its longest diameter can be accurately measured as ≥2.0 cm with chest x-ray, or as ≥1.0 cm with CT scan, CT component of a PET/CT, or MRI.
A superficial non-nodal lesion is measurable if its longest diameter is ≥1.0 cm in diameter as assessed using calipers (e.g. skin nodules) or imaging. In the case of skin lesions, documentation by color photography, including a ruler to estimate the size of the lesion, is recommended.
A malignant lymph node is considered measurable if its short axis is ≥1.5 cm when assessed by CT scan (CT scan slice thickness recommended to be no greater than 5 mm).

Non-Measurable Disease—
All other lesions (or sites of disease) are considered non-measurable disease, including pathological nodes (those with a short axis ≥1.0 to <1.5 cm). Bone lesions, leptomeningeal disease, ascites, pleural/pericardial effusions, lymphangitis cutis/pulmonis, inflammatory breast disease, and abdominal masses (not followed by CT or MRI), are considered as non-measurable as well.
Note: ‘Cystic lesions’ thought to represent cystic metastases can be considered as measurable lesions, if they meet the definition of measurability described above. However, if non-cystic lesions are present in the same patient, these are preferred for selection as target lesions. In addition, lymph nodes that have a short axis <1.0 cm are considered non-pathological (i.e., normal) and should not be recorded or followed.

### Guidelines for Evaluation of Measurable Disease

Measurement Methods:
All measurements should be recorded in metric notation (i.e., decimal fractions of centimeters) using a ruler or calipers.
The same method of assessment and the same technique must be used to characterize each identified and reported lesion at baseline and during follow-up. For patients having only lesions measuring at least 1 cm to less than 2 cm must use CT imaging for both pre- and post-treatment tumor assessments.
Imaging-based evaluation is preferred to evaluation by clinical examination when both methods have been used at the same evaluation to assess the antitumor effect of a treatment.
Acceptable Modalities for Measurable Disease:
   Conventional CT and MRI: This guideline has defined measurability of lesions on CT scan based on the assumption that CT slice thickness is 5 mm or less. If CT scans have slice thickness greater than 5 mm, the minimum size for a measurable lesion should be twice the slice thickness.
   As with CT, if an MRI is performed, the technical specifications of the scanning sequences used should be optimized for the evaluation of the type and site of disease. The lesions should be measured on the same pulse sequence. Ideally, the same type of scanner should be used and the image acquisition protocol should be followed as closely as possible to prior scans. Body scans should be performed with breath-hold scanning techniques, if possible.
   PET-CT: If the site can document that the CT performed as part of a PET-CT is of identical diagnostic quality to a diagnostic CT (with IV and oral contrast), then the CT portion of the PET-CT can be used for RECIST measurements and can be used interchangeably with conventional CT in accurately measuring cancer lesions over time.
   Chest X-ray: Lesions on chest x-ray are acceptable as measurable lesions when they are clearly defined and surrounded by aerated lung. However, CT scans are preferable.
   Physical Examination: For superficial non-nodal lesions, physical examination is acceptable, but imaging is preferable, if both can be done. In the case of skin lesions, documentation by color photography, including a ruler to estimate the size of the lesion, is recommended.
   FDG-PET: FDG-PET scanning is allowed to complement CT scanning in assessment of progressive disease [PD] and particularly possible ‘new’ disease. A ‘positive’ FDG-PET scanned lesion is defined as one which is FDG avid with an update greater than twice that of the surrounding tissue on the attenuation corrected image; otherwise, an FDG-PET scanned lesion is considered ‘negative.’ New lesions on the basis of FDG-PET imaging can be identified according to the following algorithm:
      Negative FDG-PET at baseline with a positive FDG-PET at follow-up is a sign of PD based on a new lesion.
         No FDG-PET at baseline and a positive FDG-PET at follow-up:
         If the positive FDG-PET at follow-up corresponds to a new site of disease confirmed by CT, this is PD.
         If the positive FDG-PET at follow-up is not confirmed as a new site of disease on CT at the same evaluation, additional follow-up CT scans (i.e., additional follow-up scans at least 4 weeks later) are needed to determine if there is truly progression occurring at that site. In this situation, the date of PD will be the date of the initial abnormal FDG-PET scan.
         If the positive FDG-PET at follow-up corresponds to a pre-existing site of disease on CT that is not progressing on the basis of the anatomic images, it is not classified as PD.
   Measurement at Follow-up Evaluation:
      A subsequent scan must be obtained not less than 4 weeks following initial documentation of an objective status of either complete response (CR) or partial response (PR).
      In the case of stable disease (SD), follow-up measurements must have met the SD criteria at least once after study entry at a minimum interval of not less than 6-8 weeks (see Section 11.44).
      The cytological confirmation of the neoplastic origin of any effusion that appears or worsens during treatment when the measurable tumor has met criteria for response or stable disease is mandatory to differentiate between response or stable disease (an effusion may be a side effect of the treatment) and progressive disease.
Measurement of Effect
   Target Lesions & Target Lymph Nodes
      Measurable lesions up to a maximum of 5 lesions, representative of all involved organs, should be identified as “Target Lesions” and recorded and measured at baseline. These lesions can be non-nodal or nodal, where no more than 2 lesions are from the same organ and no more than 2 malignant nodal lesions are selected.
      Note: If fewer than 5 target lesions and target lymph nodes are identified (as there often will be), there is no reason to perform additional studies beyond those specified in the protocol to discover new lesions.
      Target lesions and target lymph nodes should be selected on the basis of their size, be representative of all involved sites of disease, but in addition should be those that lend themselves to reproducible repeated measurements. It may be the case that, on occasion, the largest lesion (or malignant lymph node) does not lend itself to reproducible measurements in which circumstance the next largest lesion (or malignant lymph node) which can be measured reproducibly should be selected.
      Baseline Sum of Dimensions (BSD): A sum of the longest diameter for all target lesions plus the sum of the short axis of all the target lymph nodes will be calculated and reported as the baseline sum of dimensions (BSD). The BSD will be used as reference to further characterize any objective tumor response in the measurable dimension of the disease.
      Post-Baseline Sum of the Dimensions (PBSD): A sum of the longest diameter for all target lesions plus the sum of the short axis of all the target lymph nodes will be calculated and reported as the post-baseline sum of dimensions (PBSD). If the radiologist is able to provide an actual measure for the target lesion (or target lymph node), that should be recorded, even if it is below 0.5 cm. If the target lesion (or target lymph node) is believed to be present and is faintly seen but too small to measure, a default value of 0.5 cm should be assigned. If it is the opinion of the radiologist that the target lesion or target lymph node has likely disappeared, the measurement should be recorded as 0 cm.
      The minimum sum of the dimensions (MSD) is the minimum of the BSD and the PBSD.
      Non-Target Lesions & Non-Target Lymph Nodes
         Non-measurable sites of disease (Section 11.22) are classified as non-target lesions or non-target lymph nodes and should also be recorded at baseline. These lesions and lymph nodes should be followed in accord with 11.433

### Treatment/Follow—Up Decision at Evaluation of Patient

Patients who are CR, PR, or SD will continue treatment per protocol for a maximum of 36 cycles. After 36 cycles, patients will go to event monitoring. Patients who develop PD while receiving therapy will go to the event-monitoring phase. Patients who go off protocol treatment for reasons other than PD will go to the event-monitoring phase per Section 18.0. Patients who develop non-CNS PD at any time should go to event monitoring. These patients should be treated with alternative chemotherapy if their clinical status is good enough to allow further therapy. Event monitoring is every 3 months (±14 days) for 5 years after registration. If the patient is still alive 5 years after registration, no further follow up is required.

A patient is deemed ineligible if after registration, it is determined that at the time of registration, the patient did not satisfy each and every eligibility criteria for study entry. The patient may continue treatment off-protocol at the discretion of the physician as long as there are no safety concerns, and the patient was properly registered. The patient will go directly to the event-monitoring phase of the study (or off study, if applicable). If the patient received treatment, all data up until the point of confirmation of ineligibility must be submitted. Event monitoring will be required.

A patient is deemed a major violation if protocol requirements regarding treatment in cycle 1 of the initial therapy are severely violated that evaluability for primary end point is questionable. All data up until the point of confirmation of a major violation must be submitted. The patient will go directly to the event-monitoring phase of the study. The patient may continue treatment off-protocol at the discretion of the physician as long as there are no safety concerns, and the patient was properly registered. Event monitoring will be required per Section 18.0 of the protocol.

A patient is deemed a cancel if he/she is removed from the study for any reason before any study treatment is given.

### Binimetinib (MEK162, ARRY-438162, ONO-7703)

Background: Binimetinib is an orally bioavailable, selective and potent MEK1 and MEK 2 inhibitor. As a MEK inhibitor, this compound has the potential to benefit patients with advanced cancers by inhibiting the MAPK (mitogen-activated protein kinases) pathway.

Formulation: Binimetinib drug product is supplied as film-coated tablets in a dose strength of 15 mg. The film coated-tablets consist of binimetinib, colloidal silicon dioxide/silica colloidal anhydrous; croscarmellose sodium; lactose monohydrate; magnesium stearate; microcrystalline cellulose/cellulose, microcrystalline; and a commercial film coating. The tablet is ovaloid biconvex (capsule shaped), yellow to dark yellow in color. Binimetinib tablets can be constituted in 3:1 (v/v) Ora Sweet®/water at 1 mg/mL binimetinib concentration to provide an easy to swallow oral suspension.

Preparation and storage: Binimetinib film-coated tablets should not be stored above 25° C. and should be protected from light. Tablets are packaged in plastic bottles acceptable for pharmaceutical use.

Administration: Binimetinib is administered twice daily with water, approximately 12 hours apart with or without meals. Tablets should be swallowed whole and should not be chewed.

Pharmacokinetic information—Absorption: The pharmacokinetics of binimetinib are characterized by moderate to high variability, accumulation of approximately 1.5-fold, and steady state concentrations reached within 15 days. The human ADME study CMEK162A2102 indicated that approximately 50% of binimetinib dose was absorbed. Distribution: Binimetinib is more distributed in plasma than blood. The blood-to-plasma concentration ratio of binimetinib in humans is 0.718. It is highly bound to plasma proteins (humans: 97.2%). Metabolism: The primary metabolic pathways include glucuronidation (up to 61.2% via UGT1A1), N-dealkylation (up to 17.8% via CYP1A2 and CYP2C19) and amide hydrolysis. Excretion: The excretion route was 31.7% of unchanged binimetinib in feces and 18.4% in urine. Estimated renal clearance of unchanged binimetinib was 6.3% of total dose.

Potential Drug Interactions: Overall, the risk for binimetinib to be a cause of or be affected by significant drug-drug interactions is predicted to be low. However, given the predominant role of UGT1A1 in the metabolism of binimetinib, and because the effect of a UGT1A1 inhibitor or inducer has not been evaluated in a formal clinical study, special consideration should be taken for co-administration of drugs that are UGT1A1 inhibitors or inducers, and administration of binimetinib to patients with low UGT1A1 activity. Binimetinib has been shown to be a substrate for P-gp and BCRP in vitro. The impact of P-gp/BCRP inhibitors on the PK of binimetinib in vivo is unknown; therefore, it is recommended that P-gp and BCRP inhibitors are dosed with caution.

Known potential toxicities—Very Common (≥10%)—diarrhea, nausea, vomiting, fatigue, peripheral edema, increased AST, increased blood creatine phosphokinase, dermatitis acneiform, dry skin, pruritus, rash, decreased ejection fraction. Common (≥1%-<10%)—chorioretinopathy, dry eye, macular edema, retinal detachment, retinal vein occlusion, retinopathy, serous retinal damage, blurred vision, reduced visual acuity, visual impairment, abdominal pain, constipation, dyspepsia, gastroesophageal reflux disease, asthenia, facial edema, edema, malaise, pyrexia, folliculitis, paronychia, pustular rash, increased ALT, increased amylase, increased blood alkaline phosphatase, increased blood creatinine, increased GGT, lipase increased, arthralgia, muscular weakness, myalgia, dizziness, dysgeusia, epistaxis, alopecia, xerosis, nail disorder, palmar-plantar erythrodysesthesia syndrome, eczema, erythema, erythematous rash, papular rash, macular rash, maculo-papular rash, skin fissures, hypertension, neutropenia, a pneumonitis. Uncommon (≥0.1-<1%)— anemia, left ventricular dysfunction, eye edema, gastritis, gastrointestinal hemorrhage, colitis, general physical health deterioration, infection, skin infection, cellulitis, erysipelas, irregular heart rate, hypoglycemia, musculoskeletal pain, rhabdomyolysis, dropped head syndrome, ageusia, pulmonary embolism, xeroderma, follicular rash, pruritic rash, deep vein thrombosis, hypertensive crisis, hypotension.

### Encorafenib (LGX818, ONO-7702, Braftovi®)

Background: Encorafenib is a potent and selective ATP-competitive inhibitor of BRAF V600-mutant kinase.

Formulation: The encorafenib drug product is supplied as a hard gelatin capsule in dosage strengths of 75 mg. The dosage forms for each strength have identical formulations which are packaged in different colored capsules: 75 mg capsule (FMI): Size #00 hard gelatin capsules; flesh opaque cap and white opaque body, with the markings “NVR” or stylized “A” on the cap and “LGX 75 mg” on body.

The capsules consist of encorafenib drug substance, copovidone, poloxamer 188, succinic acid, microcrystalline cellulose, colloid silicon dioxide, crospovidone, and magnesium stearate of vegetable origin.

Preparation and storage: Encorafenib hard gelatin capsules should not be stored above 25° C. and should be protected from moisture. Capsules are packaged in plastic bottles acceptable for pharmaceutical use and should not be repackaged at the site.

Administration: Encorafenib capsules are intended for oral administration with water; capsules should be swallowed whole and should not be chewed. Encorafenib capsules may also be opened and the powder mixed with sweetened applesauce; the soft food preparation is intended for oral administration with water. Encorafenib can be administered without regard to food.

Pharmacokinetic information—Absorption: At least 86% of the dose is absorbed. Metabolism: N-dealkylation is the primary metabolic pathway; CYP3A4 (primary), CYP2C19, and CYP2D6 (minor) contribute to total oxidative clearance in human liver microsomes. Half-life elimination: 6.32 hours (range 3.74 to 8.09 hours). Excretion: Feces (39%); urine (47.2%)

Potential Drug Interactions: Since encorafenib is mainly metabolized by CYP3A, the co-administration of CYP3A inducers might decrease the exposure of encorafenib in clinical practice. Thus, long term co-administration of strong and moderate inducers of CYP3A with encorafenib should be avoided. Clinical results from a dedicated DDI study with encorafenib and CYP3A inhibitors indicated concomitant administration of encorafenib with strong or moderate CYP3A inhibitors may increase encorafenib plasma concentration. If co-administration with strong or moderate CYP3A inhibitors cannot be avoided, dose reduction of encorafenib may be warranted. Since no clinical data are available, caution should be used for co-administering substrates of CYP3A4 and UGT1A1. Based on in vitro transporter studies, encorafenib can potentially inhibit the transporters P-gp, BCRP, OATP1B1, OATP1B3, OAT1, OAT3 and OCT2 at clinical concentrations. Co-administration of encorafenib with drugs that are substrates for these enzymes and/or transporters may alter the exposure of the co-administered medication.

Known potential toxicities—Very Common (≥10%): Gastrointestinal: diarrhea; Metabolism and nutrition: decreased appetite; Nervous system: peripheral neuropathy; Psychiatric: insomnia; Skin and subcutaneous: hair loss, dry skin, hyperkeratosis, pruritis, Palmar-plantar erythrodysesthesia syndrome, palmoplantar keratoderma, erythema; Vascular: flushing. Common (≥1%-<10%): Blood and lymphatic: anemia; Cardiac: tachycardia; Ear and labyrinth: vertigo; Eye: iridocyclitis; Gastrointestinal: nausea, vomiting, abdominal pain, constipation, dyspepsia, stomatitis; General: asthenia, fatigue, pyrexia, xerosis, chills, face edema, peripheral edema; Immune system: hypersensitivity; Investigations: AST and ALT increased, blood alkaline phosphatase increased, blood creatinine increased, gamma-glutamyltransferase increased, amylase increased, lipase increased, electrocardiogram QT prolonged; Metabolism and nutrition: dehydration, hyponatremia; Musculoskeletal and connective tissue: arthralgia, musculoskeletal pain, myalgia, muscle spasms, muscular weakness; Neoplasms benign, malignant and unspecified: keratoacanthoma, melanocytic nevus, skin papilloma, squamous cell carcinoma, dysplatic nevus, malignant melanoma; Nervous system: facial paralysis, facial paresis, ageusia, dysgeusia, dysesthesia, hyperesthesia, neuralgia; Renal and urinary: acute kidney injury, renal failure; Skin and subcutaneous: rash, photosensitivity reaction, skin exfoliation, skin hyperpigmentation. Uncommon (≥0.1%-<1%)—Eye: uveitis; Gastrointestinal: pancreatitis; Metabolism and nutrition: hyperglycemia; Musculoskeletal and connective tissue: back pain, pain in extremity; Neoplasms benign, malignant and unspecified: acanthoma, basal cell carcinoma; Nervous system: hypoaesthesia; Skin and subcutaneous: drug eruption, urticarial.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A method of treating pancreatic cancer in a subject in need thereof, comprising
administering one or more MAPK pathway inhibitors to the subject, wherein the subject has a mutation in one or more genes in the MAPK signaling pathway.

2. The method of claim 1, wherein the subject has KRAS wild type.

3. The method of claim 1, wherein the mutation is in BRAF.

4. The method of claim 1, wherein the mutation is selected from:
(a) BRAF V600E,
(b) oncogenic fusion of BRAF and another gene,
(c) non-V600 mutation, insertion or deletion, or
(d) a mutation other than (a)-(c) with an additional driver mutation.

5. The method of claim 1, wherein the mutation is BRAF N486_P490del.

6. The method of claim 1, wherein the subject has a mutation in BRAF, and the one or more MAPK pathway inhibitors is a BRAF inhibitor, a MEK inhibitor or both.

7. The method of claim 1, wherein the one or more MAPK pathway inhibitors is a MEK inhibitor.

8. The method of claim 7, wherein the MEK inhibitor is trametinib, cobimetinib, binimetinib, selumetinib, or mirdametinib.

9. The method of claim 7, wherein the MEK inhibitor is Cobimetinib, Binimetinib, or Trametinib.

10. The method of claim 1, wherein one or more MAPK pathway inhibitors is dabrafenib mesylate and trametinib dimethyl sulfoxide, cobimetinib fumarate, binimetinib, binimetinib and encorafenib, trametinib dimethyl sulfoxide, selumetinib sulfate, LNP-3794, dabrafenib mesylate, panitumumab and trametinib dimethyl sulfoxide, HL-085, mirdametinib, MK-2206 and selumetinib sulfate, trametinib dimethyl sulfoxide and uprosertib, ATI-450, ATR-002, CKI-27, CS-3006, durvalumab and selumetinib sulfate, E-6201, FCN-159, SHR-7390, TQB-3234, ABM-1383, ATR-004, ATR-005, ATR-006, EBI-1051, KZ-001, OTS-514, OTS-964, Small Molecule to Inhibit MKK4 for Acute Liver Failure and Chronic Liver Disease, Small Molecules to Inhibit Mitogen Activated Protein Kinase Kinase for Oncology, or SNR-1611.

11. The method of claim 1, wherein the one or more MAPK pathway inhibitors is a BRAF inhibitor.

12. The method of claim 11, wherein the BRAF inhibitor is dabrafenib mesylate and trametinib dimethyl sulfoxide, sorafenib tosylate, binimetinib and encorafenib, dabrafenib mesylate, encorafenib, vemurafenib, sorafenib tosylate, dabrafenib mesylate, panitumumab and trametinib dimethyl sulfoxide, hydroxychloroquine and sorafenib tosylate, lifirafenib maleate, TAK-580, BAL-3833, belvarafenib, CKI-27, LUT-014, LXH-254, RXDX-105, TQB-3233, XP-102, UB-941, ABM-1310, AFX-1251, APL-102, ARI-4175, AZ-304, BGB-3245, INU-152, LYN-204, PV-103, REDX-05358, or SJP-1601.

13. The method of claim 11, wherein the BRAF inhibitor is Dabrafenib, Vemurafenib, Encorafenib, Lifirafenib, belvarafenib, or sorafenib tosylate.

14. The method of claim 11, wherein the BRAF inhibitor is Vemurafenib, Dabrafenib or Encorafenib.

15. The method of claim 1, wherein the one or more MAPK pathway inhibitors is Dabrafenib and trametinib, Vemurafenib and cobimetinib, Trametinib, or Vemurafenib.

16. The method of claim 1, wherein the one or more MAPK pathway inhibitor is Binimetinib and Encorafenib.

17. The method of claim 1, wherein the one or more MAPK pathway inhibitors is Vemurafenib and the method further comprises administering carboplatin, paclitaxel or both.

18. The method of claim 1, wherein the one or more MAPK pathway inhibitors is Trametinib and the method further comprises administering Pembrolizumab.

19. The method of claim 1, further comprising administering one or more chemotherapy drugs, one or more PD-1 inhibitors or PD-L1 inhibitors, both a chemotherapy drug and a PD-1 inhibitor, or both a chemotherapy drug and a PD-L1 inhibitor.

20. The method of claim 1, wherein the one or more MAPK pathway inhibitor is Binimetinib and Encorafenib, wherein the mutation is BRAF V600E, and the pancreatic cancer is BRAF V600E mutated pancreatic ductal adenocarcinoma (PDAC).

21. The method of claim 1, wherein the subject's pancreatic cancer has progressed on at least one line of therapy for metastatic disease or wherein the subject is intolerant of at least one line of therapy for metastatic disease.

22. The method of claim 1, wherein the subject's pancreatic cancer has recurred with metastatic disease less than or equal to 12 weeks of completion of neoadjuvant or adjuvant systemic chemotherapy, or wherein the subject has locally advanced pancreatic cancer whose pancreatic cancer progressed to metastatic disease less than or equal to 12 weeks after completion of systemic chemotherapy, or wherein the subject's pancreatic cancer has recurred with metastatic disease less than or equal to 12 weeks of completion of systemic chemotherapy.
